(19) United States
(12) Patent Application Publication
(10) Pub. No.: US 2020/0023206 A1
(43) Pub. Date: Jan. 23, 2020

(54) SYSTEMS AND METHODS FOR ULTRASOUND MODULATION OF NEURONS

(71) Applicant: THE TRUSTEES OF COLUMBIA UNIVERSITY IN THE CITY OF NEW YORK, New York, NY (US)

(72) Inventors: Elisa E. KONOFAGOU, New York, NY (US); Ellen A. LUMPKIN, New York, NY (US); Yoshichika BABA, New York, NY (US); Chi-Kun TONG, Bronx, NY (US); Benjamin HOFFMAN, New York, NY (US); Matthew E. DOWNS, New York, NY (US); Danny M. FLOREZ PAZ, Manhattan, NY (US)

(73) Assignee: THE TRUSTEES OF COLUMBIA UNIVERSITY IN THE CITY OF NEW YORK, New York, NY (US)

(21) Appl. No.: 16/357,127

(22) Filed: Mar. 18, 2019

Related U.S. Application Data
(63) Continuation of application No. PCT/US2017/052310, filed on Sep. 19, 2017.
(60) Provisional application No. 62/396,553, filed on Sep. 19, 2016.
(60) Provisional application No. 62/396,930, filed on Sep. 20, 2016.
(60) Provisional application No. 62/440,170, filed on Dec. 29, 2016.

(51) Int. Cl. A61N 7/00; A61B 8/00; G01N 33/487

(52) U.S. Cl. CPC A61N 7/00; A61N 2007/0026; G01N 33/48728; A61B 8/4494

(57) ABSTRACT

Systems for modulating one or more neurons using focused ultrasound (FUS) include a transducer mount, a recording chamber disposed at an angle relative the transducer mount and configured to contain the one or more neurons within the recording chamber, an ultrasound transducer disposed on the transducer mount to provide an ultrasound stimulus having one or more ultrasound parameters to the one or more neurons, and a processor configured to adjust the one or more ultrasound parameters to produce one or more action potentials from the one or more neurons in response to the ultrasound stimulus, the one or more action potentials corresponding to one or more of a pain or sensation response, a pain or sensation suppression, or neural control of organ function induced by the one or more neurons.

### CROSS-REFERENCE TO RELATED APPLICATIONS

This application is a continuation-in-part of International Patent Application Serial No. PCT/US17/052310, filed on Sep. 19, 2017 which claims priority to U.S. Provisional Patent Application Nos. 62/440,170, filed Dec. 29, 2016; 62/396,930, filed Sep. 20, 2016; and 62/396,553, filed Sep. 19, 2016; each of which is incorporated by reference herein in its entirety.

### STATEMENT REGARDING FEDERALLY-SPONSORED RESEARCH

This invention was made with government support from the Department of Defense/Defense Advanced Research Projects Agency (DOD/DARPA) under Grant No. HR0011-15-2-0054 and National Institute of Neurocritical Disorders and Stroke (NINDS) under Grant No. F31NS105449. The Government has certain rights in the invention.

### BACKGROUND

Ultrasound is a versatile technology that is used in many different fields such as imaging, chemical processes, and therapeutics. Ultrasound is a widespread technique for monitoring fetal development or cardiac abnormalities, and can be employed as a therapeutic treatment for procedures that require non-invasive, target specific, and temporally efficient procedures. Certain techniques can utilize the ability of ultrasound to have thermal, mechanical or a combined thermal/mechanical effect. For example, focused ultrasound (FUS) can involve concentrating multiple intersecting beams of ultrasound on a target region using an acoustic lens. Given the precision and non-invasive nature of the technique, certain FUS-related methods have been utilized for the treatment of a variety of diseases including prostate cancer and uterine fibroids.

Certain therapeutic ultrasound techniques utilizing FUS can be effective at stimulating, or inhibiting neuronal activity in both the central nervous system (CNS) and peripheral nervous system (PNS). For example, FUS can open the blood-brain barrier and thus can facilitate the diffusion of drug molecules into brain tissue. Moreover, FUS can modulate neuronal activity by stimulating specific regions in the CNS and FUS can stimulate or inhibit the PNS due to either thermal or mechanical effects of FUS.

Peripheral neuropathy, a condition that can develop as a result of damage to the PNS, can cause symptoms including tingling in extremities and inaccurate touch sensations. Certain treatments for peripheral neuropathy can be either non-invasive and non-target specific or invasive and targeted. For example, transcutaneous electrical nerve stimulation (TENS) is one such treatment that can be used for stimulating peripheral nerves. However, while TENS can be non-invasive, it can also be non-specific, e.g., by targeting regions of nerves around a specific damaged peripheral nerve.

Therapeutic ultrasound can provide for both targeted and non-invasive treatment of peripheral neuropathy, eliminating the potential side effects of invasive therapies while being capable of targeting specific peripheral nerves for treatment. Additionally, FUS systems can be relatively inexpensive and portable, allowing treatment to be applied to patients at clinics or at home by the patient trained to operate the system.

Accordingly, there remains a need in the art for improved techniques for targeted, specific, and non-invasive treatment options that can modulate neurons, for example, sensory or motor neurons, for the treatment of neuropathy or other disorders.

### SUMMARY

The presently disclosed subject matter utilizes ultrasound technology to excite or inhibit neurons, including but without limitation, sensory neurons, motor neurons, or other cells. Example systems for modulating one or more neurons using focused ultrasound (FUS) include a transducer mount, a recording chamber disposed at an angle relative the transducer mount and configured to contain the one or more neurons therein, an ultrasound transducer disposed on the transducer mount to provide an ultrasound stimulus having one or more ultrasound parameters to the neurons, and a processor. The processor can be configured to adjust the ultrasound parameters to produce one or more action potentials from the neurons in response to the ultrasound stimulus. The action potentials can correspond to one or more of a pain or sensation response, a pain or sensation suppression, or neural control of organ function induced by the one or more neurons.

Example methods for modulating one or more neurons using focused ultrasound (FUS) include providing a recording chamber at an angle relative a transducer mount, inserting the neurons within the recording chamber, generating with an ultrasound transducer an ultrasound stimulus having one or more ultrasound parameters to the neurons, and adjusting the ultrasound parameters. The ultrasound parameters can be adjusted to produce one or more action potentials from the neurons in response to the ultrasound stimulus.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter. Moreover, the principles of the disclosed subject matter can be implemented in various configurations and are not intended to be limited in any way to the specific embodiments presented herein.

### BRIEF DESCRIPTION OF THE DRAWINGS

FIG. 1 is a diagram illustrating ultrasound evoked action potentials in sensory neurons.

FIGS. 2A-2B are diagrams illustrating an exemplary embodiment of a system for ultrasound neuromodulation according to the disclosed subject matter. FIG. 2A is a diagram illustrating an exemplary nerve for use with the disclosed subject matter. FIG. 2B is a diagram illustrating an exemplary embodiment of a system for ultrasound neuromodulation according to the disclosed subject matter, including for applying ultrasound to the exemplary nerve of FIG. 2A ex vivo.

FIGS. 3A-3H are diagrams illustrating additional details of sensory neurons according to the disclosed subject matter. FIG. 3A is a diagram illustrating ultrasound stimulation of neuronal receptive fields in the ex vivo nerve preparation of FIGS. 2A-2B.

FIGS. 3B-3D are diagrams illustrating exemplary ultrasound parameters according to the disclosed subject matter. FIGS. 3B-3D each illustrate exemplary recordings of Ab (FIG. 3B), Ad (FIG. 3C), and C-fibers (FIG. 3D), with the top line illustrating the ultrasound stimulus trace; the middle line illustrating representative traces of ultrasound-evoked action potentials, and the bottom image illustrating ultrasound stimulus duration compared with ultrasound pressure. The scale indicates the probability of ultrasound-evoked action potential firing. FIG. 3E is a diagram illustrating a mechanical threshold compared with an ultrasound pressure threshold of sensory receptive fields in the ex vivo nerve preparation (N=83 fibers). The broken line represents an estimated threshold to define LTMRs (≤1 mN) and nociceptors (>1.0 mN).

FIG. 3F is a diagram illustrating ultrasound inhibition of mechanically-activated neuronal responses in the ex vivo nerve preparation of FIGS. 2A-2B. FIG. 3F illustrates the whole saphenous nerve (bottom) pulsed with ultrasound for 4 seconds before identified receptive fields are stimulated by a computer controlled mechanical indenter (top). FIG. 3G is a diagram illustrating ultrasound inhibition of neuronal responses. FIG. 3G illustrates a representative example of ultrasound inhibition techniques (Ad, CV=7.67 m/s), with the top representing a force trace of the mechanical stimulus, the middle line representing the ultrasound stimulus trace, and the bottom image representing a raster plot of neuronal responses. One trial included 4 seconds of ultrasound, a 1-second interval, and a 5-second ramp and hold mechanical stimulus. Trials were sequentially applied with a 50-second inter-trial interval. The scale indicates ultrasound pressure applied with either 1% or 5% duty cycle, as indicated. The dotted line indicates long-lasting but reversible suppression of mechanically evoked responses. FIG. 3H is a diagram illustrating ultrasound modulation of mechanically evoked responses. FIG. 3H illustrates a representative example from FIGS. 3B-3D. The mean firing rate of mechanically evoked responses compared to pressure of the ultrasound stimulus applied directly before mechanical stimulation at 1% or 5% duty cycle, as indicated.

FIGS. 4A-4B are diagrams together illustrating another exemplary embodiment of a system for ultrasound neuromodulation according to the disclosed subject matter, including for applying ultrasound to sensory neurons in vitro. FIGS. 4A-4B illustrate the integration of an ultrasound device into an inverted electrophysiology rig. FIG. 4A illustrates an exemplary chamber including an ultrasound transducer adapter and a clamping ring to seal the culture coverslip. FIG. 4B illustrates the exemplary chamber of FIG. 4A disposed in the stage of an inverted microscope with space to allow transmitted light to pass the cells and such that the cells can be patched on the coverslip.

FIG. 5 is a diagram illustrating noise and stimulus intensity. The color cord from thin to thick illustrates amplitude-intensity. Left labels indicate amplitude intensity, duty cycle, and measured output stimulus duration. The Trig. line represents the timing of ultrasound initiation. The horizontal bar US illustrates the 7.5 msec stimulus input duration. The measured output duration is indicated by the labels on the left.

FIG. 6 is a diagram illustrating shield and noise source. From bottom to top: the first five signals (orange) represent normal location; the next five signals (blue) represent normal location with copper shielding; the next five signals (pink) represent normal location and ringer removed; the next five signals (green) represent outside of receptive field; and the last five signals (brown) represent outside of skin.

FIG. 7 is a diagram illustrating ultrasound response in sensory afferents for large unit (Aβ) CV=12.4 m/sec (top); middle unit (Aδ) CV=8.7 m/sec (middle); and small unit (C fiber) CV=about 2.0 m/sec (bottom).

FIG. 8 is a diagram illustrating ultrasound elicited action potentials in intact tissue. Representative traces of ultrasound elicited action potentials are shown. Single action potentials are shown in response to ultrasound stimulation in Aβ (10.1 m/s; top), Aδ (7.0 m/s; middle), and C fibers (0.3 m/s; bottom). The horizontal bar below a portion of each action potential indicates the duration of the ultrasound stimulus (1 ms, 100% duty cycle, 35 MPa, 3.57 MHz).

FIG. 9 is a diagram illustrating action potential firing probability at various ultrasound intensities.

FIG. 10 is a diagram illustrating ultrasound stimulation and electrical stimulation elicited action potentials with similar conduction velocities (CVs) for purpose of comparison. Each point represents a single neuronal unit (n=11). The line illustrates a linear regression.

FIG. 11 is a diagram illustrating levels of tissue damage from repeated ultrasound stimulation. Each image indicates hematoxylin and eosin (HE) staining of skin sections after repeated ultrasound stimulation at various pressures (pressures: 15 MPa, 35 MPa, 65 MPa; duration 7 ms; duty cycle: 90%; frequency: 3.57 MHz; stimulus number: 64; stimulus interval: 10 s). Arrowheads indicate approximate stimulus location.

FIG. 12 is a diagram illustrating exemplary sensory neurons activated by ultrasound stimulation. Different stimulation intensities were delivered at 3.57 MHz, 98% duty cycle for 5 ms (5 pulses, 1 KHz).

FIG. 13 is a diagram illustrating exemplary ultrasound responses recorded in a sensory neuron under a voltage clamp. The stimulus (3.1 MHz, ˜3 MPa) was delivered for 0.95 ms. Repeated stimulus presentations elicited different degrees of responses from the same neuron.

FIG. 14 is a diagram illustrating exemplary ultrasound responses from a sensory neuron under a current clamp. Ultrasound (3.1 MHz, ˜3 MPa) was delivered at three different pulse frequencies (1, 4, and 7 KHz, 20% duty cycle). The upper panel shows three traces under three corresponding stimulating paradigms shown in the lower panel. An action potential and excitatory membrane potentials were induced by repeated ultrasound stimuli.

FIGS. 15A-15K are schematic diagrams illustrating an exemplary system for in vitro stimulation of sensory neurons in accordance with an embodiment of the presently disclosed subject matter. FIG. 15A is a perspective view illustrating a chamber for ultrasound stimulation. FIG. 15B is a bottom view of the chamber. FIG. 15C is a side view of the chamber. FIG. 15D is a top view of the chamber. FIG. 15E is a perspective view of a screw system for use with the chamber. FIG. 15F is a bottom view of the screw system. FIG. 15G is a side view of the screw system. FIG. 15H is a schematic view illustrating exemplary dimensions of the ultrasound mount and chamber, for purpose of illustration only and not limitation. FIG. 15I is a schematic view illustrating exemplary dimensions of the ultrasound mount and chamber, for purpose of illustration only and not limitation. FIG. 15J is a schematic view illustrating exemplary dimensions of the ultrasound mount, for purpose of illustration only and not limitation. FIG. 15K is a schematic view illustrating exemplary dimensions of the chamber, for purpose of illustration only and not limitation.

FIGS. 16A-16B are diagrams illustrating another exemplary system for in vitro stimulation of sensory neurons in accordance with an embodiment of the presently disclosed subject matter, including a laser positioning system to target the ultrasound beam focus. FIG. 16A is a perspective view illustrating integration of a confocally aligned laser module into the back of the ultrasound transducer (Chamber A+laser). FIG. 16B is a schematic diagram showing high-intensity ultrasound for physical identification of the ultrasound beam focal area (left, light spot, light arrow). The etched area aligns with the laser beam (right, dark spot, dark arrow).

FIGS. 17A-17B are diagrams illustrating another exemplary system for in vitro stimulation of sensory neurons in accordance with an embodiment of the presently disclosed subject matter. FIG. 17A is a perspective view illustrating a two-component laser positioning system (Chamber B+laser). FIG. 17B is a pair of images illustrating laser identified ultrasound beam focus (top) and ultrasound etched beam focus (bottom).

FIG. 18 is a diagram illustrating additional details of the laser positioning system for ultrasound stimulation. For purpose of illustration and comparison, chamber A is the original chamber design. Chamber B+laser is the two-component laser positioning system. Ultrasound threshold for activation was measured on cells in each chamber design. *p=0.038, **p=0.0024, one-way ANOVA, Bonferroni post-hoc, means±SD.

FIGS. 19A-19B are diagrams illustrating exemplary ultrasound-induced whole-cell currents recorded from a HEK cell at −70 mV (FIG. 19A) and +30 mV (FIG. 19B) in Chamber B+laser. Ultrasound stimulation parameters, as embodied herein, are 3.1 MHz, 1.28 MPa, 25 μs, 1 stimulus, 30 s intervals.

FIGS. 20A-20D are diagrams illustrating ruthenium red (RR) on ultrasound (US)-induced currents. FIG. 20A illustrates representative examples of ultrasound-induced currents before (control), during (ruthenium red), and after (washout) RR treatment (black, first stimulus; blue, second; red, third). As embodied herein, cell was held at −90 mV. Ultrasound stimulation parameters were 3.1 MHz, 1.6 MPa, 25 μs, 1 stimulus, 30 s intervals. FIG. 20B illustrates additional representative examples of ultrasound-induced currents with RR treatment. As embodied herein, cell was held at −90 mV. Ultrasound stimulation parameters were the same as FIG. 20A, except the pressure was 1.27 MPa. FIG. 20C illustrates a summary of the drug effects of ruthenium red on ultrasound-induced currents (N=5 cells). As embodied herein, responses to three stimulus presentations per condition were averaged and normalized to the mean amplitude in the control condition for each cell. Experiments were done at −70 mV or −90 mV holding potential. FIG. 20D illustrates HEK cells grouped into two populations according to ultrasound activation thresholds and ruthenium red sensitivity. As embodied herein, cells with higher ultrasound activation thresholds showed higher ruthenium sensitivity than those with lower ultrasound activation thresholds.

FIGS. 21A-21B are diagrams illustrating exemplary effects of sodium substitution with N-methyl-d-glucamine (NMDG) on ultrasound-induced currents. FIG. 21A illustrates representative examples of ultrasound-induced currents in control solution (Ringers), NMDG-Ringers (without sodium), and in Ringers washout (black, first stimulus; blue, second; red, third). As embodied herein, the cell was held at −90 mV. Ultrasound stimulation parameters were 3.1 MHz, 1.88 MPa, 25 μs, 1 stimulus, 30 s intervals. FIG. 21B illustrates NMDG substitution on ultrasound-induced currents (N=5 cells). As embodied herein, responses to three stimulus presentations per condition were averaged and normalized to the mean amplitude in the control condition for each cell. Cells were held at −90 mV. Four cells showed reduced responses, one cell showed an increased response following NMDG replacement. Replacement of sodium with NMDG in the medium did not show significant effect on ultrasound-induced currents (un-normalized data, P=0.16 for paired students t test, two-tailed).

FIGS. 22A-22I are diagrams illustrating exemplary effects of ultrasound on action potentials from sensory neurons. FIG. 22A illustrates an exemplary system for evoking action potentials. FIG. 22B illustrates additional details of an exemplary laser guided targeting of receptive fields shown in 2201 of FIG. 22A. FIG. 22C illustrates an exemplary immersion cone for use with the exemplary system of FIG. 22A. FIG. 22D illustrates an exemplary driving signal produced by the system of FIG. 22A. FIG. 22E illustrates an exemplary X-Y ultrasound beam profile without (left) and with (right) the cone. FIG. 22F illustrates an exemplary X-Z beam profile measured from the cone tip (z=0). FIG. 22G illustrates exemplary FUS evoked action potentials measured by the system of FIG. 22A. FIG. 22H illustrates an exemplary aggregate FUS parameter-probability space of neurons recorded by the system of FIG. 22A. FIG. 22I illustrates an exemplary transform of FIG. 22H into total sonication energy.

FIGS. 23A-23H are diagrams illustrating distinct classes of mechanosensory neurons excited by FUS. FIG. 23A illustrates exemplary touch- and ultrasound-evoked action potentials from afferents categorized by mechanosensory classes. FIG. 23B illustrates exemplary space exploration data at fixed intensities (460 W/cm2, left) and (390-540, ˜30 W/cm2 steps, right) with fixed duration (0.75 ms). FIG. 23C illustrates exemplary space exploration data at fixed intensities (155 W/cm2, left) and (45-340, ˜55 W/cm2 steps, right) with fixed duration (0.75 ms). FIG. 23D illustrates exemplary space exploration data at fixed intensities (460 W/cm2, left) and (45-340, ˜55 W/cm2 steps, right) with fixed duration (0.75 ms). FIG. 23E illustrates exemplary space exploration data at fixed intensities (220 W/cm2, left) and (45-340, ˜55 W/cm2 steps, right) with fixed duration (0.75 ms). FIG. 23F illustrates exemplary space exploration data at fixed intensities (220 W/cm2, Right) and (45-340, ˜55 W/cm2 steps, right) with fixed duration (0.75 ms). FIG. 23G illustrates an exemplary aggregate FUS parameter-probability space representation of neurons recorded, separated by mechanosensory class. FIG. 23H illustrates an exemplary transform of the data from (F) into total sonication energy.

FIGS. 24A-24B are diagrams illustrating exemplary details of action potentials. FIG. 24A illustrates an exemplary action potential failure (indicated by asterisks) that can occur with increased FUS intensity. FIG. 24′B illustrates an exemplary action potential failure (indicated by asterisks) that can occur with increased FUS duration.

FIGS. 25A-25E are diagrams illustrating exemplary action potentials evoked by nerve trunk FUS stimulation. FIG. 25A illustrates an exemplary system for evoking nerve system with trunk FUS stimulation. FIG. 25B illustrates exemplary recorded action potentials from Aβ, Aδ, and C-fibers. FIG. 25C illustrates an exemplary system for comparing receptive field and nerve trunk FUS stimulation. FIG. 25D illustrates an exemplary comparison of FUS-evoked action potentials elicited by receptive field stimulation and nerve trunk stimulation. FIG. 25E illustrates an exemplary comparison of sonication energy thresholds (e.g., having at least 50% probability of eliciting an action potential) from FUS stimulation targeted to receptive fields or nerve trunks from the same fiber.

FIGS. 26A-26C are diagrams illustrating exemplary action potentials evoked by millisecond latencies of FUS. FIG. 26A illustrates an exemplary comparison of exemplary FUS-evoked to electrically evoked action potentials from the same Aβ-SA fiber. FIG. 26B is a diagram illustrating log-transformed FUS-evoked versus electrically evoked action potential latencies. FIG. 26C is a diagram illustrating exemplary differences between FUS-evoked and electrically evoked action potential latencies from each neuron.

FIGS. 27A-27D are diagrams illustrating exemplary FUS stimulated-action potentials through mechanically activated ion channel Piezo2. FIG. 27A illustrates an exemplary system for stimulating action potential through mechanically activated ion channel. FIG. 27B illustrates exemplary electrically-, mechanically-, and FUS-evoked action potentials from control and Cdx2Cre;Piezo2fl/fl mice. FIGS. 27C-27D illustrate cumulative response plots for mechanical thresholds (FIG. 27C) and FUS thresholds (FIG. 27D) from control and Cdx2Cre;Piezo2fl/fl mice.

### DETAILED DESCRIPTION

The presently disclosed subject matter relates to the use of ultrasound technology to excite or inhibit neurons in mammals. Ultrasound technology can enable non-invasive stimulation of inaccessible areas, such as deep brain tissue, and can be used, for example and without limitation, as a therapeutic tool and as a technique to determine neuronal mechanisms. The response properties of ultrasound neuromodulation in peripheral sensory afferents can be used to develop improved bioelectronic therapeutics. For example and without limitation, ultrasound stimulation can directly generate action potentials (APs) in peripheral neurons through activation of mechanosensitive ion channels. Non-invasive ultrasound-based therapeutics can be used to treat neurological diseases such as chronic pain and neuropathies. Additionally, the present disclosure provides techniques for high-throughput screening of mechanosensitive ion-channel-specific pharmacology as well as mechanosensitive ion channels.

The disclosed subject matter provides systems and methods for modulation of neurons using ultrasound. Although certain embodiments, for purpose of illustration only, describe neuromodulation of sensory neurons, the systems and techniques described herein can be used to modulate any neurons, including but without limitation, sensory, motor or cardiac neurons. Such modulation of neurons, as described further herein, can be used for excitation or inhibition of sensory response for pain treatment or suppression, and/or excitation or inhibition of other neurons to provide motor or organ control, such as control of cardiac functions. In certain embodiments, the systems and methods can use focused ultrasound to modulate neurons in the peripheral nervous system.

In certain aspects, the present disclosure provides methods that can excite or inhibit sensory neurons, for example, in mammals, using focused ultrasound. Applying focused ultrasound to sensory neurons can elicit action potentials within the neurons. For example, focused ultrasound can activate mechanosensitive ion channels within the neurons. The neurons can include Aβ, Aδ, and/or C fibers. In certain embodiments, the neurons or other mammalian cells can be obtained from Hek 293T, HeCaT, iPSc (induced pluripotent stem cells), and DRG neuron cells. The presently disclosed methods and systems can be non-invasive, and can be used in the treatment of neurological diseases, including for chronic pain and peripheral neuropathies.

In certain aspects, the sensory neurons can be ex vivo. For example, and as embodied herein, an ex vivo preparation can include skin-saphenous nerves. Methods can include applying focused ultrasound to the ex vivo preparation. In certain embodiments, methods for preparing an ex vivo preparation can include dissecting the skin and saphenous nerves of a mammal. For example, after dissection, the skin sample can be placed epidermis-side-up in a chamber with optionally buffered interstitial fluid. The skin sample can be maintained in a temperature-controlled environment. A nerve sample can be maintained in mineral oil and placed in a recording chamber for ultrasound stimulation.

In certain embodiments, the focused ultrasound can have a frequency of about 3.57 MHz, a duration of about 1 ms, and/or a duty cycle of about 100%. The intensity of the focused ultrasound can be from about 2 MPa to about 50 MPa. In particular embodiments, an intensity of from about 15 MPa to about 45 MPa can be applied. Alternatively, the intensity of the focused ultrasound can be from about 1.1 MPa to about 8.3 MPa. In particular embodiments, an intensity of from about 3.2 MPa to about 5 MPa. These intensities can elicit action potentials within the neurons. In certain embodiments, each neuron can fire a single action potential. Alternatively, each neuron can fire a train of action potentials. In certain embodiments, the focused ultrasound can be applied to the receptive fields of one or more neurons.

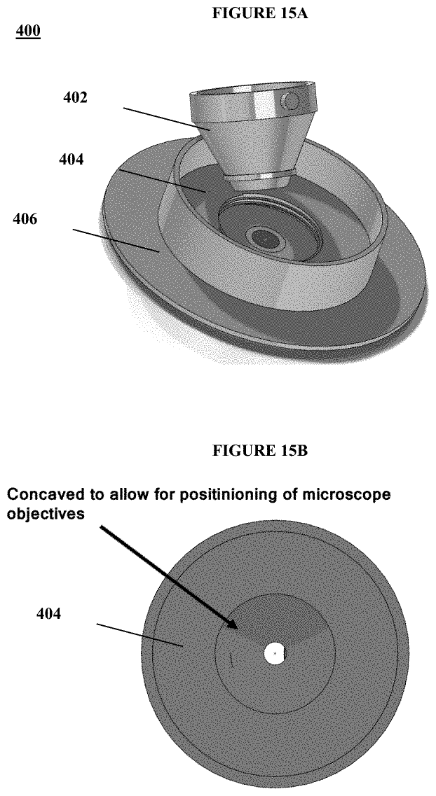

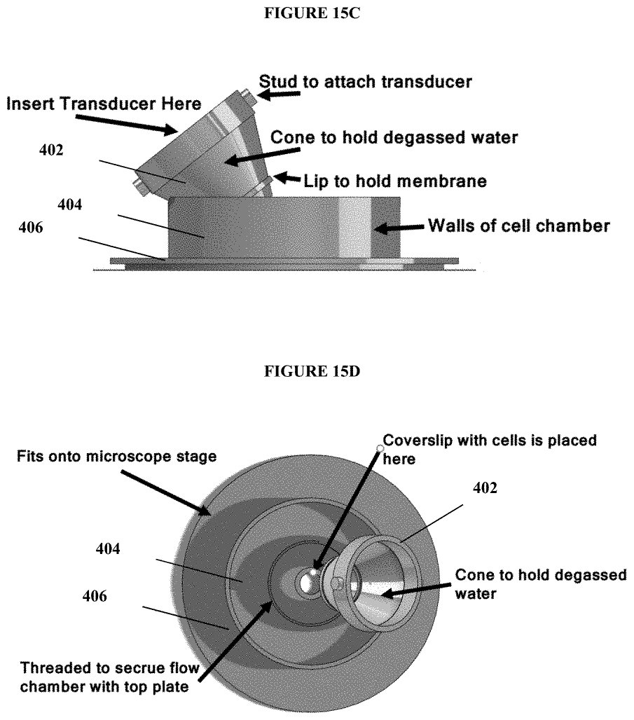

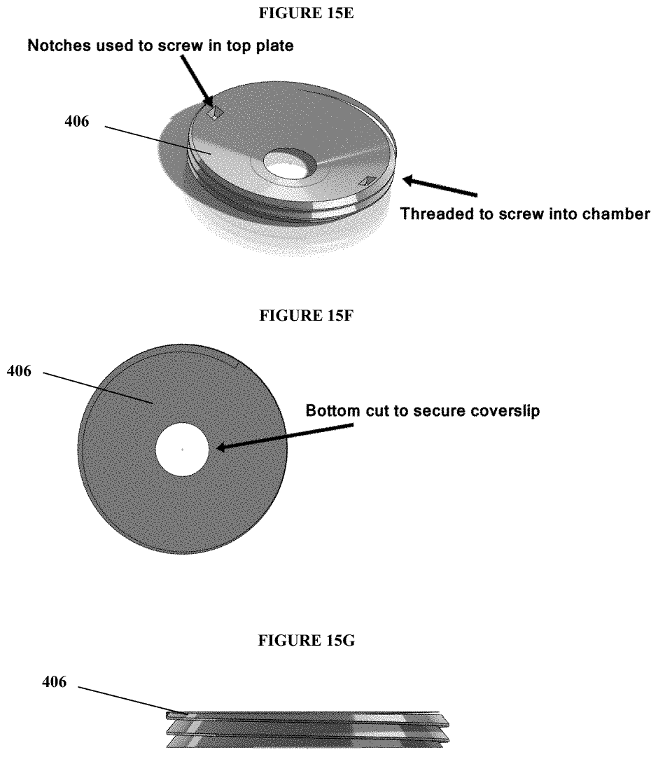

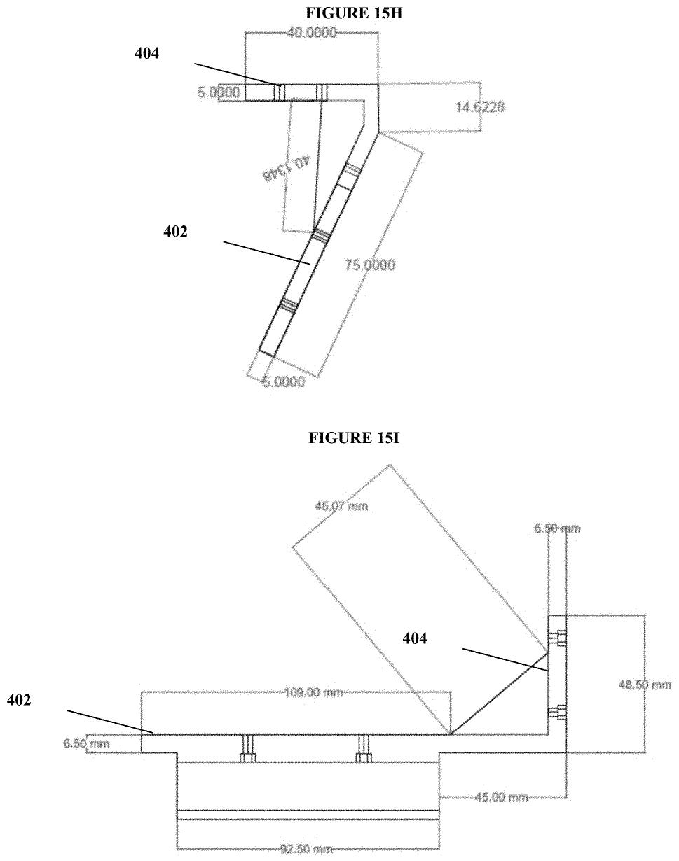

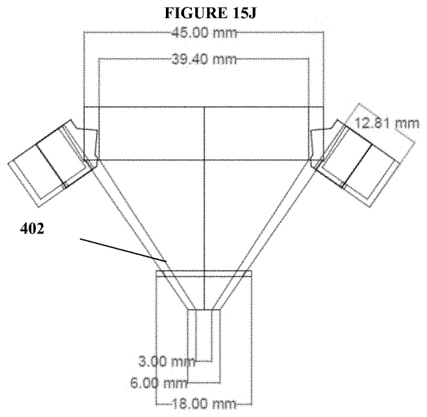

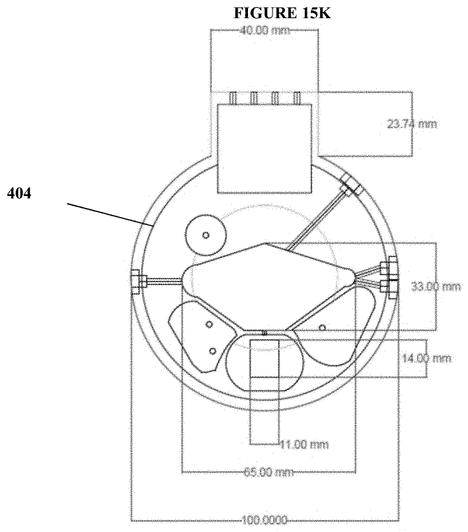

In certain other aspects, the neurons can be in vitro. The present disclosure further provides systems for the in vitro stimulation of sensory neurons. Systems can include a cellular imaging plate system having a chamber and incorporated with an ultrasound transducer. (See FIGS. 15A-15K). The system can be used in conjunction with various microscope objectives. In certain embodiments, the system can include a Mylar substrate. In certain embodiments, the system can further include a screw system configured to secure the Mylar substrate and prevent leakage from the chamber to the microscope objectives. As embodied herein, the chamber can further include a water bladder coupled to the transducer. Such systems are suitable for high throughput screening of sensory neurons.

Methods of applying focused ultrasound to in vitro dissociated sensory neurons are also provided. Neuronal responses can be measured using an inverted microscope capable of detecting electrophysiology. Pipette resistance can range from about 4 MΩ to about 7 MΩ. The method can include first establishing a gigaohm seal, then breaking the seal and applying vacuum to obtain a seal resistance of from about 200 MΩ to about 800 MΩ. For example and not limitation, the method can include measuring current and membrane capacitance. In certain embodiments, the resting membrane potential can be measured and the firing rate can be calculated. In certain embodiments, the ultrasound stimulation can be performed using a 3.1 MHz transducer, with a 1 ms stimulus duration with 25% duty cycle. The pipette solution can contain one or more of CsCl, NaCl, MgCl₂, CaCl₂), EGTA, MgATP, HEPES, and TEA. The extracellular solution can contain one or more of NaCl, KCl, HEPES, D-glucose, MgCl₂, and CaCl₂). The pH of the extracellular solution can be adjusted to a neutral pH, e.g., a pH of about 7.4.

In certain other aspects, the sensory neurons can be in vivo. Methods can include applying focused ultrasound to in vivo sensory neurons and measuring an electrophysiological response therefrom. In certain embodiments, the in vivo sensory neurons can be within the peripheral nervous system. For example, and not limitation, the sensory neurons can be cutaneous nerves, such as saphenous nerves. Additionally or alternatively, and without limitation, the sensory neurons can be vagus nerves.

Additionally, methods for neuromodulation of sensory neurons can further include imaging calcium within one or more sensory neurons while applying focused ultrasound to determine the level of cytoplasmic calcium within the neurons. An increase in calcium levels can be indicative of activation of mechanosensitive ion channels. Methods can further include imaging calcium after applying focused ultrasound to determine the level of cytoplasmic calcium after ultrasound treatment. In certain embodiments, the level of cytoplasmic calcium can be approximately equal to the level of cytoplasmic calcium prior to ultrasound treatment. Methods can further include measuring electrophysiology, for example, action potentials, in voltage-clamp mode and/or current-clamp mode.

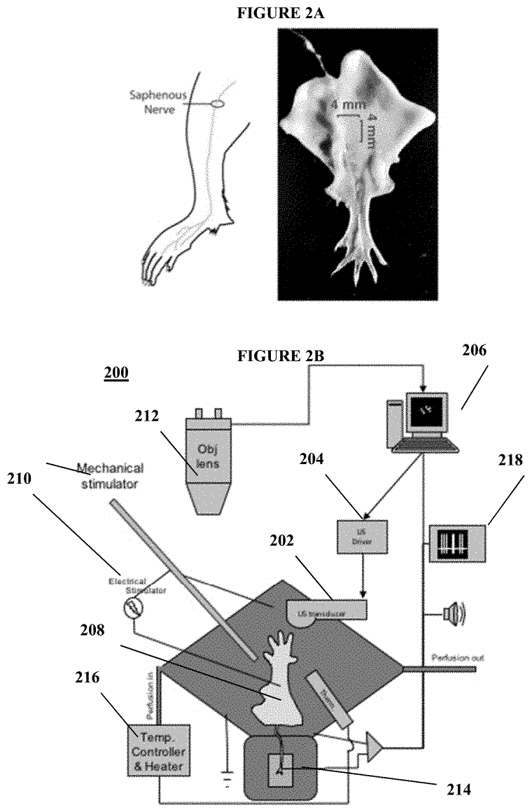

Ultrasound neuromodulation can be evaluated in neurons in ex vivo and in vitro preparations. As shown in FIG. 2, system 200 for ex vivo ultrasound neuromodulation of sensory neurons includes an ultrasound transducer 202, which can be driven by an ultrasound driver 204 in communication with a processor 206. For purpose of illustration and not limitation, using an ex vivo skin-saphenous nerve preparation 208, extracellular electrophysiological recordings can be performed in conjunction with ultrasound application (as embodied herein, using ultrasound transducer 202 at 3.57 MHz, 1 ms, 100% duty cycle, 2-50 MPa) to produce ultrasound-activated APs 218 firing in mechanosensory afferents of adult mice (n=32). As embodied herein, the ultrasound threshold for driving APs can be 15-45 MPa.

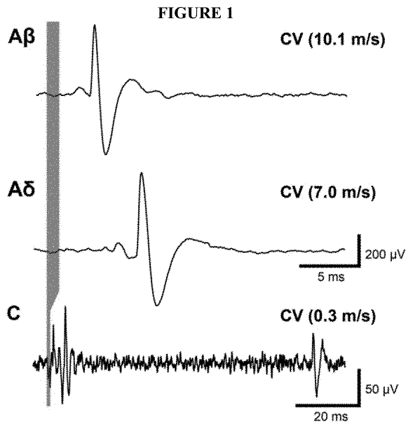

For further example, and not limitation, ex vivo extracellular electrophysiological recordings can be performed in conjunction with ultrasound application (as embodied herein, using ultrasound transducer 202 at 3.57 MHz, 1 ms, 100% duty cycle, 1.1-8.3 MPa) to produce ultrasound-activated AP firing in mechanosensory afferents of adult mice (n=32). As embodied herein, the ultrasound threshold for driving APs can be 3.2-5 MPa. Ultrasound can elicit APs 218 in multiple classes of peripheral neurons, including touch receptors and nociceptors. The latency of AP firing after ultrasound stimulation can depend at least in part on sensory neuron type and ultrasound intensity. Additionally, firing latency can decrease with increasing ultrasound. AP waveform and latencies can be similar to electrically evoked responses in the same receptive field. Ultrasound stimulation can elicit action potentials 218 in all mechanoreceptive sensory neurons, including without limitation, Aβ, Aδ, and C fibers. Under certain ultrasound stimulation parameters, neurons can fire a single action potential 218 in response to ultrasound. In response to certain other ultrasound stimulation parameters, for example and as embodied herein in the upper end of pressure ranges employed, ultrasound can elicit trains of action potentials 218. With reference to FIG. 1, the responses illustrated provide a parameter space for performing peripheral ultrasound stimulation and for determining the mechanisms of ultrasound induced neuronal firing. The action potentials can be indicative of one or more of a pain or sensation response, pain or sensation inhibition, or neural control of organ function.

Referring now to FIGS. 2A-2B, in accordance with aspects of the disclosed subject matter, the present disclosure provides methods for obtaining ex vivo skin-saphenous recordings. For purpose of illustration and not limitation, with reference to FIG. 2A, ultrasound-evoked responses in the skin can be recorded after dissecting the hindlimb skin and saphenous nerve. As embodied herein, the skin was placed epidermis-side-up in a custom chamber and perfused with carbogen-buffered synthetic interstitial fluid (SIF) maintained at 32° C. using a temperature controller (e.g., model TC-344B, Warner Instruments). Referring now to FIG. 2B, the nerve was kept in mineral oil in a recording chamber, teased apart and placed onto a silver recording electrode connected with a reference electrode to a differential amplifier (e.g., model 1800, A-M Systems). The extracellular signal was digitized using a digitizer 214, embodied herein using a DT304 A/D board (DataWave Technologies) and recorded using Sci-Works Experimenter software (DataWave Technologies). System 200 can optionally include one or more stimulators 210, such as a mechanical stimulator and/or electrical stimulator, to provide additional stimulation to the nerve 208, as described herein. System 200 can optionally include an optical detector, such has microscope 212, to allow for visual detection and recording of the nerve 208, as described further herein. System 200 can optionally include a temperature controller 216, such as a heater, which can include one or more temperature sensors, to control the thermal environment around nerve 208.

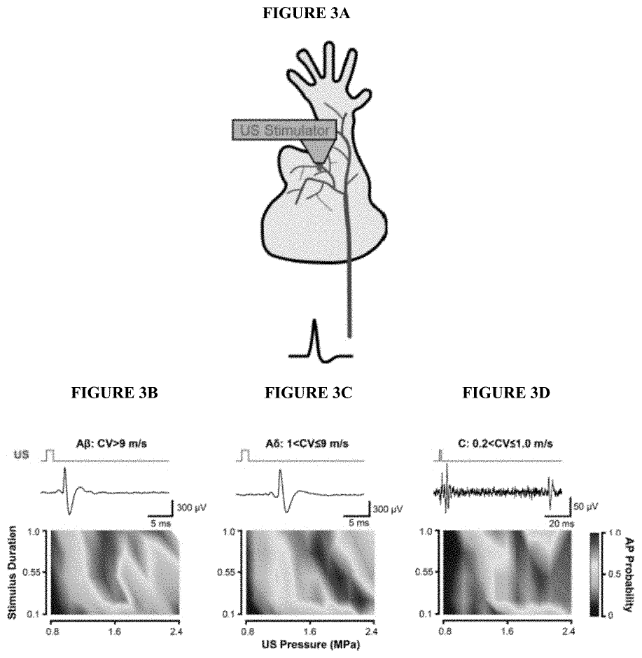

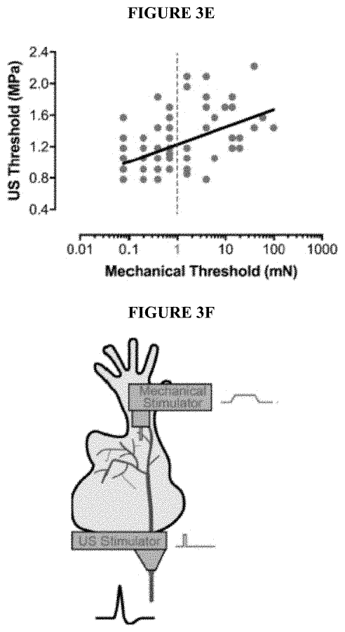

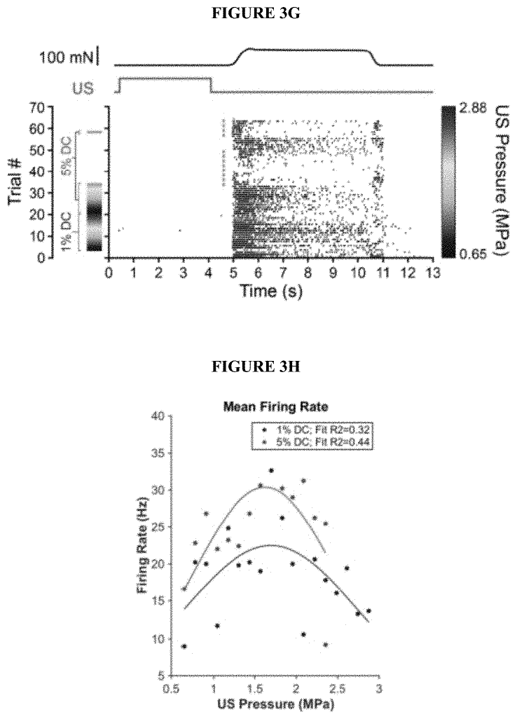

For purpose of illustration and confirmation of the disclosed subject matter, with reference to FIG. 3A, ultrasound can elicit single action potentials from Aβ, Aδ, and C-fibers in the intact skin-saphenous nerve preparation 208. As described further herein, ultrasound parameters to selectively activate or excite functionally distinct subtypes of peripheral neurons can be determined. Different ultrasound stimulus parameters can excite sensory neurons. FIGS. 3B-3D illustrate exemplary probabilities that a selected ultrasound parameter set can evoke an action potential in a peripheral neuron. To determine action potential probability, for example and not limitation, and as embodied herein, each ultrasound parameter set was presented 4-10 times, and probability was defined as the fraction of stimuli that elicited an action potential. As embodied herein, ultrasound stimulus duration (e.g., 0.1-1.0 ms, 0.1-0.5 ms steps) and ultrasound pressure (e.g., 0.8-2.4 MPa, 0.15-0.3 MPa steps) were varied, while ultrasound frequency (e.g., 3.57 MHz) and inter-stimulus interval (e.g., 10 s) remained fixed. As such, stimulus was provided using 116 different ultrasound parameter combinations in Aβ (N=48 units), Aδ (N=58 units), and C-fibers (N=18 units). Fibers were classified based on action potential conduction velocity, obtained though electrical stimulation of receptive fields.

Referring still to FIGS. 3B-3D, Aβ fibers (FIG. 3B) were activated most robustly at mid-range ultrasound pressures, and over a wide range of ultrasound stimulus durations. Aδ fibers (FIG. 3C) were activated over a wider range of ultrasound pressures and stimulus durations as compared to Aβ fibers, but were not activated at combinations of high ultrasound pressures and high stimulus durations. C-fibers (FIG. 3D) were activated over a wide range of ultrasound pressures and stimulus durations. The ultrasound parameter space topography thus differed significantly between Aβ, Aδ, and C-fibers, indicating that functionally distinct subtypes of peripheral neurons can have unique response profiles to ultrasound stimulation. In this manner, specific ultrasound parameter sets can be defined to selectively excite different classes of sensory neurons in intact tissue. As shown in FIGS. 3B-3D, Aβ fibers can be selectively activated by mid-range pressures and stimulus durations, Aδ fibers can be selectively activated by high-range pressures and low-range stimulus durations, and C-fibers can be selectively activated by high-range pressures. Pulsed ultrasound at different frequencies can also be utilized to achieve selective activation.

The classification of sensory neurons (Aβ, Aδ, and C-fibers) can depend at least in part on conduction velocity, as well as, for example in the case of mechanoreceptors, mechanical threshold for activation. Aβ and a group of Aδ fibers can have the lowest threshold for activation (referred to herein as low-threshold mechanoreceptors or LTMRs), while C and a separate group of Aδ fibers can have higher thresholds for activation, and thus can be considered nociceptors. As shown in FIGS. 3B-3D, the ultrasound pressure threshold for activation was lowest in Aβ fibers and highest in C-fibers. With reference to FIG. 3E, as embodied herein, mechanical threshold was evaluated in fibers by stimulation of receptive fields with calibrated Von Frey fibers. As shown in FIG. 3E, mechanical threshold positively correlates (as embodied herein, semilog linear model, slope=0.22, P<0.0001, R2=0.25) with ultrasound threshold (as embodied herein, 1-ms stimulus duration). As such, ultrasound pressure can be used to differentially activate LTMRs and nociceptors in intact tissue.

For purpose of illustration and confirmation of the disclosed subject matter, referring now to FIG. 3F, inactivation of peripheral neurons using ultrasound is illustrated. As described further herein, ultrasound parameters to selectively inhibit functionally distinct subtypes of peripheral neurons can be determined. To determine if ultrasound stimulation of neurons can inhibit neuronal responses, an additional stimulator 210 (FIG. 2B), embodied herein as a computer-controlled mechanical stimulator, was utilized along with the ultrasound transducer 202. In this configuration, receptive fields of identified sensory neurons can be stimulated, while either simultaneously or consecutively stimulating the same sensory afferent at a more proximal location in the saphenous nerve, as shown for example in FIG. 3F.

For purpose of illustration and not limitation, and as embodied herein, identified sensory neurons in the ex vivo skin-saphenous nerve preparation 208 were stimulated with a 4-second ultrasound stimulus (as embodied herein, US frequency=3.57 MHz, US pressure=0.65-2.88 MPa, PRF=1 KHz, DC=1 or 5%), followed by a 1-second interval, and then a 5-second compressive mechanical stimulus. For excitation, as embodied herein, a single US stimulus of less than 1 ms evoked neuronal responses. For inhibition, as embodied herein, longer (>1 s), pulsed US stimuli, with low duty cycles, were effective. Such US-mechanical stimulus was sequentially applied to sensory neurons every minute (e.g., inter-trial interval=50 s), while action potentials were recorded. Ultrasound had two primary effects on action potential firing: long-lasting, reversible inhibition of mechanically evoked firing (as shown for example in FIG. 3G), or increasing/decreasing action potential firing rate (as shown for example in FIG. 3H). In three examples, significant ultrasound suppression of mechanically evoked activity lasted for more than 20 minutes, before recovering to near baseline responsiveness. By comparison, in 6 out of 9 examples, ultrasound sonication of the saphenous nerve resulted in non-reversible loss of mechanically evoked action potentials. The non-reversible nature of these events can indicate damage to neuronal tissue. As such, ultrasound sonication energy can be applied to neuronal tissue up to a certain amount before observing damage. This amount can be represented, as embodied herein, as an ultrasound stimulus with the following parameters: 3.57 MHz frequency, 4 s stimulus duration, 2.5 MPa Pressure, 1 KHz PRF, and 5% DC. The total sonication energy can be reduced by reducing stimulus duration and duty cycle (e.g., 3.57 MHz frequency, 2 s stimulus duration, 0.65-2.4 MPa Pressure, 1 KHz PRF, and 1% DC). These results illustrate ultrasound inhibition of peripheral neuronal activity and provide an ultrasound parameter range for inhibition.

For purpose of illustration and confirmation of the disclosed subject matter, with reference to FIG. 13, in calcium imaging experiments, many neurons showed robust increases in cytoplasmic calcium that returned to baseline after stimulation, indicating that responses do not simply reflect cellular damage. As embodied herein, some desensitization to repeated stimulus presentations was observed in some neurons. Such desensitization is typical for mechanosensory responses in sensory neurons. In voltage-clamp electrophysiological recordings, ultrasound elicited transient inward currents of several hundred picoamperes. The magnitude and transient nature of these currents is consistent with activation of mechanosensitive channels. Consistent with calcium imaging experiments, desensitization to repeated stimulation was observed. In current clamp mode, ultrasound pulse trains activated action potentials, as shown for example in FIG. 13. The action potentials can be indicative of one or more of a pain or sensation response, pain or sensation inhibition, or neural control of organ function.

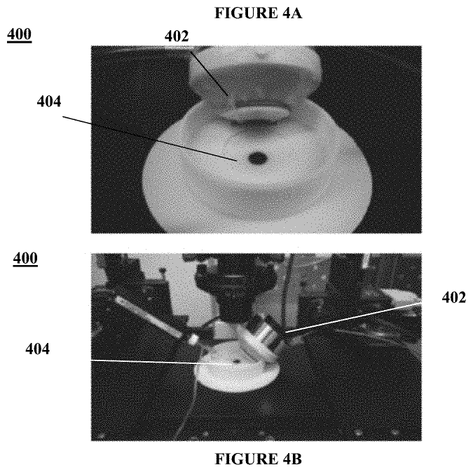

Additionally or alternatively, as embodied herein, ultrasound can be applied to in vitro dissociated sensory neurons. With reference to FIGS. 4A-4B and 15A-15K, system 400 for in vitro ultrasound neuromodulation of sensory neurons can include any or all features of system 200. Additionally or alternatively, system 400 can include a cellular imaging plate system incorporating an ultrasound transducer, which can be utilized for precise targeting of the cells. The cellular imaging plate system can include an ultrasound transducer mount 402 disposed at an angle relative a recording chamber 404. In certain embodiments, Mylar, an acoustic and visually transparent material, can form a substrate for cell attachment as an alternative to glass. Glass can create large reflections of an ultrasound wave, and thereby generate multiple wave fronts passing through the cells, which can cause confounding effects that do not exist in in vivo models. As such, the sensory neurons can be harvested and grown on Mylar coverslips. A screw system 406 can lock the Mylar in place, sealing the recording chamber 404 and preventing chamber medium from leaking out onto the microscope objectives. This recording chamber 404 also incorporates a water bladder for coupling the ultrasound waves generated from the transducer with the cell chamber. This configuration can allow for stimulation of the cells that mimics in vivo properties, while allowing for multiple imaging experiments to verify ultrasound neuromodulation (e.g., calcium imaging, patching for voltage and current clamps). In certain embodiments, this system can be modified to fit multiple ultrasound transducers with varying housing sizes and focusing distances.

In accordance with aspects of the disclosed subject matter, methods for obtaining in vitro recordings are provided. With reference to FIGS. 4A-4B, for purpose of illustration, and not limitation, as embodied herein, recording can be obtained from multiple different cell types including Hek 293T, HeCaT, iPSc (induced pluripotent stem cells), and DRG neurons. Currents can be recorded, as embodied herein, with an Axopatch 200B amplifier, a Digidata 1440A interface and a personal computer running pClamp 10 software (Axon Instruments). Pipette resistance can range from 4-7 MΩ. For example, after a gigaohm seal is established, the seal can be broken by suction and a 200-800 MΩ seal resistance can be obtained. Membrane capacitance can be measured from the decay constant during a 1 ms voltage step using pClamp software. Signals can be filtered at 10 kHz and digitized at 25 μs. Leak currents can be compensated during whole-cell recordings with a P/4 leak subtraction protocol. Junction potential can be measured for each intracellular solution used and can be compensated before each experiment. For DRG neurons, resting membrane potential can be measured and firing rate can be calculated using a serial of squared pulses increasing 20 pA each step from −120 pA to 300 pA in current clamp mode. Currents elicited by ultrasound stimulation can be recorded in the whole-cell configuration and voltage clamp mode, clamping the cells at −70 mV. Ultrasound stimulation can be performed using a 3.1 MHz transducer, with a 1 ms stimulus duration with 25% duty cycle and variable ultrasound pressures. As embodied herein, the pipette solution can contain (in mM): 120 CsCl, 10 NaCl, 1 MgCl₂, 1 CaCl₂), 10 EGTA, 2 MgATP, 10 HEPES and TEA 10 (pH 7.2, adjusted with KOH). Extracellular solution can contain (in mM): 140 NaCl, 5 KCl, 10 HEPES 10 D-glucose, 2 MgCl₂ and 2 CaCl₂). (pH 7.4, adjusted with NaOH and molarity adjusted to 325 mOsm with Sucrose). For DRG, for example and as embodied herein, the pipette solution can contain (in mM): 135 KCl, 10 NaCl, 1 MgCl₂, 1 CaCl₂), 10 EGTA, 2 MgATP, 10 HEPES and TEA 10 (pH 7.2, adjusted with KOH).

According to aspects of the disclosed subject matter, an ultrasound transducer can be integrated into an ex vivo skin nerve preparation electrophysiological recording system. For example, and as embodied herein, an integrated system can allow for precise timing of ultrasound stimulation, and yield sufficient electrical signal-to-noise (ratio=3) to resolve action potentials, for example and without limitation, from single Aβ, Aδ, and C fibers in teased peripheral nerves.

For purpose of illustration and not limitation, integration of the ultrasound device into the ex vivo rig can occur without an increase in baseline electrical noise. As embodied herein, a “comb-shaped” noise artifact was observed when the ultrasound transducer was activated. The duration of this ultrasound artifact was less than 0.5 msec, and was generally shorter than ultrasound elicited sensory spike durations. Thus, the ultrasound elicited spike amplitude can be large enough to allow for identification of spikes even within the ultrasound artifact. With reference to FIG. 5, for purpose of illustration and not limitation, and as embodied herein, the amplitude of the ultrasound artifact did not depend on ultrasound input voltage, but did depend at least in part on duty cycle and pulse duration.

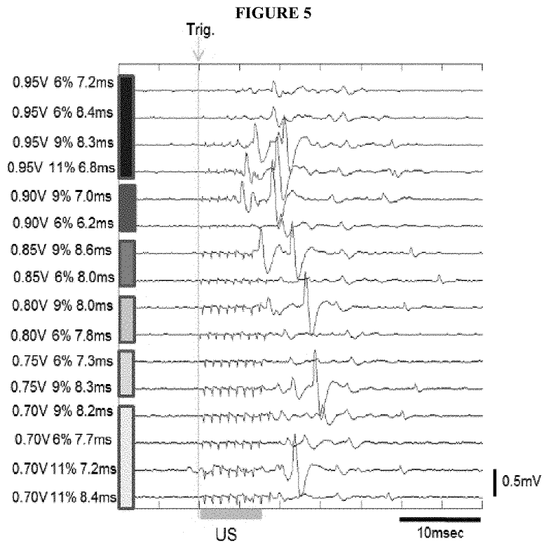

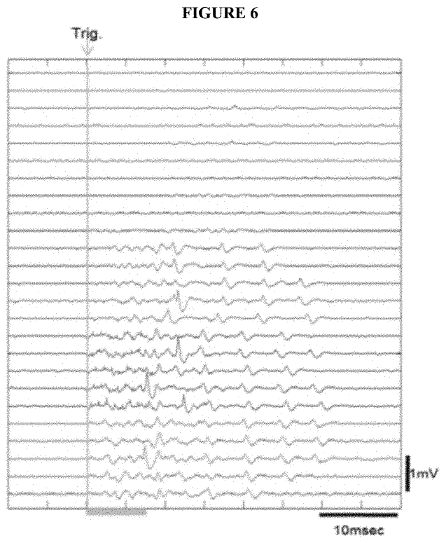

For purpose of illustration and not limitation, and as embodied herein, input voltage inversely correlates with spike latency, and duty cycle positively correlates with the probability of ultrasound elicited spikes. As such, the parameter spaces for stimulating peripheral sensory nerves can be defined. For example and without limitation, three approaches to identify the source of the ultrasound noise artifact were performed: shielding the transducer with copper, removing the solution between transducer and skin, and changing stimulus locations. As embodied herein, shielding did not reduce the noise, but instead actually enhanced the noise. Removing the solution between the transducer and skin reduced noise, but the effect was small. When the transducer was moved outside of the receptive field or to a region in the recording chamber without skin, there was no longer any observable noise. As illustrated for example in FIG. 6, the data suggests that the origin of the noise was more likely mechanical than electrical.

Referring now to FIG. 7, data has been recorded from 4 mice and more than 15 units have been resolved (as embodied herein, Aβ 4; Aδ 8; C-fiber 1; Unknown 2).

For purpose of illustration and confirmation of the disclosed subject matter, as embodied herein techniques were performed to validate that focused ultrasound can elicit action potentials from sensory neurons in the skin nerve preparation. Such techniques were performed to generate an initial dataset that explores the ultrasound parameter space that can stimulate neurons in intact skin. Such techniques can allow for phenotyping the neurophysiological properties of ultrasound-activated and ultrasound-inhibited fibers.

For purpose of illustration and without limitation, integration and activation of the ultrasound transducer in an ex vivo skin nerve preparation rig did not introduce significant noise, as discussed herein above. As embodied herein, refined stimulus and recording techniques were determined to better resolve ultrasound-elicited action potentials from Aβ, Aδ, and C fibers, and the ultrasound parameter space that elicits action potentials from Aβ, Aδ, and C fibers was shown.

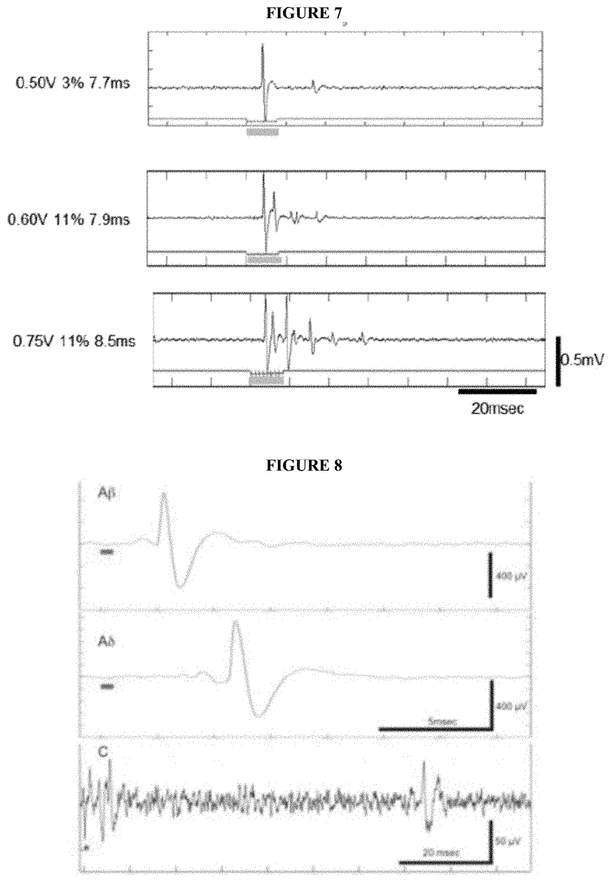

With reference to FIG. 8, for example and without limitation, and as embodied herein, ultrasound stimulation elicited action potentials in all mechanoreceptive sensory neurons (Aβ, Aδ, and C fibers) that were recorded (32/32 fibers). For the majority of ultrasound stimulation parameters, neurons fired a single action potential in response to ultrasound. However, in response to the upper end of the pressure range employed, it was occasionally observed that ultrasound elicited trains of action potentials.

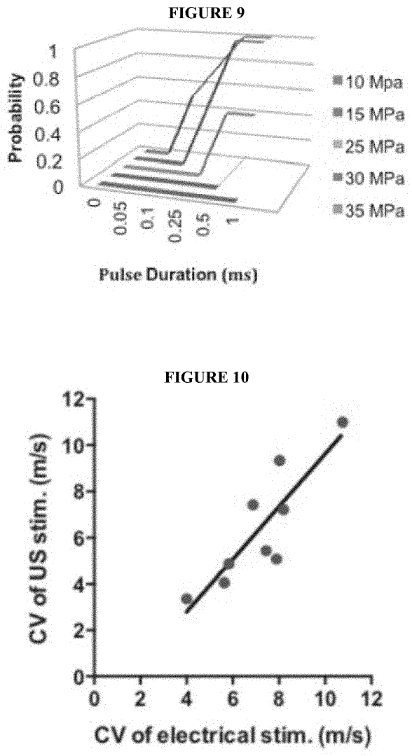

Referring now to FIG. 9, as a basis for understanding optimal ultrasound parameters, the relationship between ultrasound intensity and neuronal firing probability is illustrated. As shown, increasing ultrasound intensity, as a function of both stimulus duration and ultrasound pressure, uniformly increased the probability of action potential firing.

With reference to FIG. 10, additionally and as embodied herein, ultrasound-evoked action potentials in sensory neurons have conduction velocities that are positively correlated with electrical stimulation of the same receptive field (as embodied herein, slope=1.13, R2=0.75, P=0.0027). The latency of ultrasound evoked action potentials decreases with increasing ultrasound intensities, and as such, the precision of measuring conduction velocity of ultrasound responses has inherent variability.

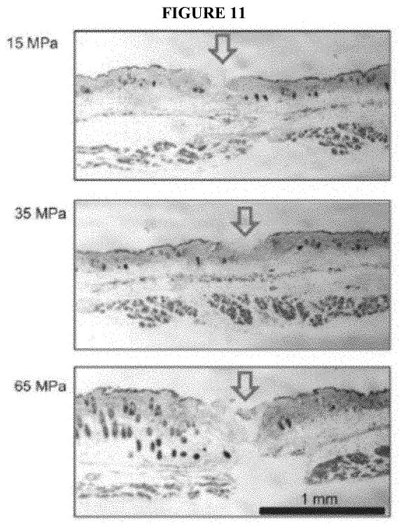

Furthermore, for purpose of illustration and not limitation, and as embodied herein, the range of ultrasound intensity that can activate neurons without damage, such as necrosis or excitotoxicity, as well as the threshold for causing cellular damage was determined. No evidence of damage was found in tissue stimulated with ultrasound pressures and durations in the physiological data reported above. With reference to FIG. 11, for purpose of comparison, as embodied herein, repeated application (64 times) of ultrasound stimuli resulted in significant structural damage to targeted tissue. As such, both intensity of ultrasound stimulation, as well as stimulus number, can be reduced or limited in order to reduce or minimize tissue damage.

In addition, for purpose of illustration and not limitation, and as embodied herein, the mechanisms of ultrasound stimulation on peripheral nerves can be determined. An ultrasound transducer was integrated into an existing in vitro recording apparatus for live-cell imaging and recordings from cells. The system was aimed to allow precise timing of ultrasound stimulation, and to yield sufficient electrical signal to noise ratio (>3) to resolve currents from single cells and neurons.

In order to integrate the ultrasound device (referred to as “Device A”) with the inverted microscope electrophysiology rig, the following techniques were performed. For example and not limitation, as embodied herein, an appropriate coverslip material was identified. Mylar was chosen at least in part because this material avoids the significant ultrasound absorption and reflection of traditional glass coverslips. No significant differences between cells cultured on glass coverslips or Mylar were observed. Additionally, and as embodied herein, a recording chamber was formed to fix an ultrasound transducer to the stage, specifically in an orientation in which the ultrasound focus is at the center of the coverslip. Further, and as embodied herein, the transducer was oriented at an angle selected to enable both transmitted light microscopy as well as physical access to the cells with a patch pipette. For example and not limitation, as embodied herein, the the angle of the recording chamber is about 25 degrees relative the transducer. For example and as embodied herein, the chamber was made with 3D printing technology. With reference to FIG. 4A, the recording chamber 404 includes a fitting ring at the bottom center that can clamp a Mylar membrane, and as such, physiological saline solution in the chamber is tightly sealed from the bottom. The recording chamber 404 is configured to allow easy access of a patch pipette to the cells without blocking the transmitted light under the microscope, as shown for example in FIGS. 4B and 15A-15D.

For example and without limitation, as embodied herein, cells can be plated on Mylar coverslips to facilitate of cellular activity after ultrasound stimulation. While the example presented here relates to plating DRG neurons, the techniques described herein can be applied to any types of cells that can be cultured on Mylar material and activated by ultrasound. For example and without limitation, other cells, such as HEK cells and iPSCs (induced pluripotent stem cells) can also be cultured on Mylar for use with the ultrasound techniques described herein. As embodied herein, to form Mylar coverslips, a single Mylar sheet was cut into smaller pieces sized to be disposed in the chamber. The coverslips were sterilized with UV and 95% alcohol for 20 minutes. The coverslips were rinsed with sterilized water, and coated with laminin (50 μg/ml) for one hour.

As embodied herein, using for example and without limitation dorsal root ganglion (DRG) or trigeminal (TG) neurons, DRGs or TGs from P45-P90 mice were dissected, collected and digested in collagenase P (7 mg in 5 ml HBSS) and 0.25% trypsin at 37° C. separately. DRG or TG neurons were neutralized with 10% horse serum containing B-27 MEM (19 mL MEM, 200 μl Penn-strep, 200 μl Vitamins, 400 μl B27) and titrated with a glass pipette, and the DRG or TG neurons were centrifuged and re-suspended in B-27 MEM medium. The cells were gently titrated with a p200 pipette to dissociate remaining clumps and were plated on the Mylar coverslips. Cell cultures were used for imaging or recording 2-36 hours following plating.

The activity of individual cells can be imaged by measuring the internal calcium activity following ultrasound stimulation. For example and without limitation, as embodied herein, for calcium imaging, DRG or other cells were incubated in a Ringer solution (in mM, 145 NaCl, 5 KCl, 10 HEPES, 10 glucose, 2 CaCl2, 2 MgCl2, pH 7.3, 325 mOsm with sucrose) containing Fura-2 AM (5 μM) and Pluronic F-127 (0.01%) for 30-45 mins. The cells were then carefully rinsed with Ringer solution 1-2 times to remove unbound Fura-2 AM. The Mylar coverslips were then placed and clamped into the bottom of the chamber. As embodied herein, an Olympus 10×/0.4 objective was used for ratiometric calcium imaging under the IX81 inverted microscope.

Additionally or alternatively, as embodied herein, the electrical signals of the cells can also be recorded using electrophysiology recording techniques, such as without limitation, whole cell patch clamp recording. Whole cell current or voltage clamp recordings were performed on these DRG neurons or other cells. Internal solution included (in mM) 120 potassium methanesulfonate, 10 KCl, 10 NaCl, 5 EGTA, 0.5 CaCl2, 10 Hepes, 2.5 MgATP, pH adjusted to 7.2 with KOH, osmolarity 280 mosmol. As embodied herein, cells were held at −70 mV for voltage clamp recording.

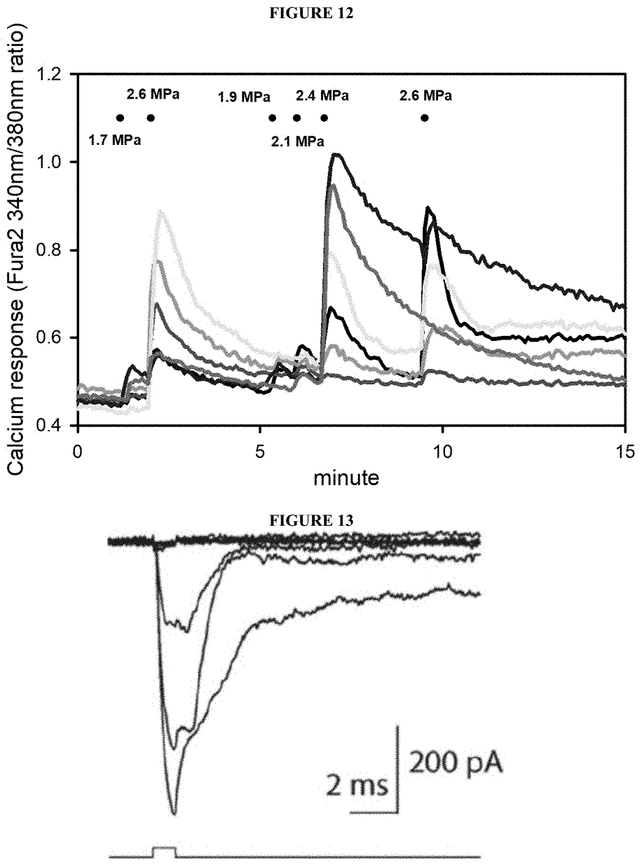

For purpose of illustration and not limitation, as embodied herein, examples were performed to determine whether integration of the ultrasound device into the rig introduces significant increase in optical or electrical noise in in vitro live cell imaging and electrophysiological recordings, respectively. Fura-2 ratiometric calcium imaging was used to monitor activation of DRG neurons. With reference to FIG. 12, neuronal responses were repeatedly elicited upon ultrasound stimulation. No optical noise was observed during ultrasound stimulation.

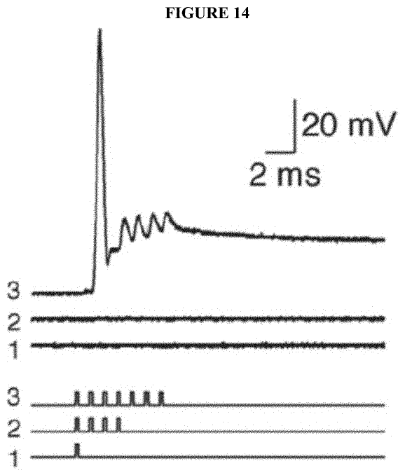

Integration of the ultrasound device into the inverted microscope did not introduce an increase in baseline electrical noise, but as embodied herein, did cause stimulus transients at the onset and offset of ultrasound pulses (e.g., 5-30 pA, typically 10-20 pA, ˜100 μs) in voltage-clamp mode. Referring now to FIG. 13, the amplitude and duration of these ultrasound transients did not interfere with neuronal signals, as described herein, which outlast the duration of the stimulus transients. In current clamp mode, with reference to FIG. 14, the noise level was <1 mV, which was insignificant compared with the magnitude of action potentials.

Activation of DRG neurons by ultrasound was further performed. Referring still to FIGS. 12-14, ultrasound elicited robust excitatory responses in sensory neurons, as measured by calcium imaging (FIG. 12), voltage-clamp recordings (FIG. 13) and current clamp recordings (FIG. 14).

In calcium imaging experiments, as embodied herein, many neurons showed robust increases in cytoplasmic calcium that returned to baseline after stimulation, indicating that responses do not simply reflect cellular damage. Some desensitization to repeated stimulus presentations was observed in some neurons (e.g., purple and pink traces in FIG. 12). Such desensitization is typical for mechanosensory responses in sensory neurons. At the upper end of the ultrasound range tested, Mylar substrates and neurons were damaged near the focal area.

In voltage-clamp mode, ultrasound elicited transient inward currents of several hundred picoamperes, as shown for example in FIG. 13. As embodied herein, the magnitude and transient nature of these currents is consistent with activation of mechanosensitive channels. Consistent with calcium imaging experiments, desensitization to repeated stimulation was observed. In current clamp mode, ultrasound pulse trains activated action potentials, as shown for example in FIG. 14.

Although Mylar coverslips were used to avoid absorption and reflection of ultrasound, electrophysiological pipettes are made of glass that can absorb ultrasound. Increased holding currents were observed following ultrasound stimuli, suggesting some leakage of the patch was generated during stimulation. Additionally, and as embodied herein, some ultrasound induced vibration of the pipette was observed when it was in the path of ultrasound propagation in solution. To reduce or minimize vibration that might lead to seal leakage, the pipette can be positioned away from the ultrasound path but the tip can be kept close the ultrasound focal area. As embodied herein, an ultrasound prototype was successfully integrated with electrophysiology, microscopy, and data acquisition equipment for in vitro live-cell imaging and cell recordings, including without limitation, with signal-to-noise ratios greater than three.

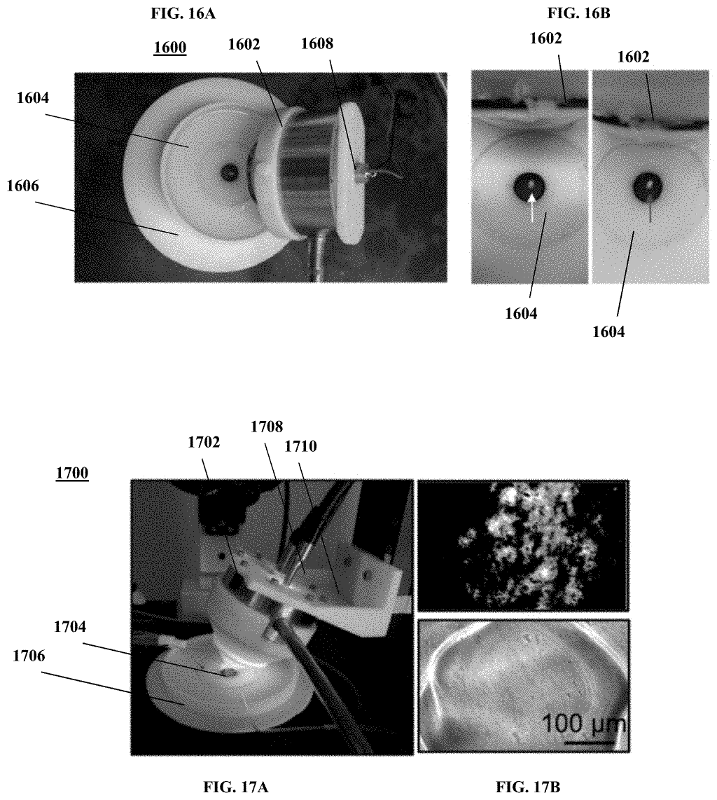

According to other aspects of the disclosed subject matter, exemplary systems and techniques to measure biophysical and pharmacological profiles of ultrasound-activated currents are provided. Such systems and techniques can be used, for example and without limitation, to implicate specific molecular ion channel entities. For purpose of illustration and not limitation, as embodied herein, pharmacological agents and ion substitution can be used to establish ion selectivity of ultrasound stimulated conductances in cell lines. Such techniques can be performed using the in vitro system 400 of FIGS. 4A-4B and 15A-15K, and which can include certain modifications as illustrated in system 1600 of FIGS. 16A-16B. System 1600 for in vitro ultrasound neuromodulation of sensory neurons can include any or all features of systems 200 or 400. Certain modifications can enhance the reliability and efficiency of ultrasound stimulation of mammalian cells. For example and without limitation, as embodied herein, in system 1600, both the ultrasound transducer mount 1602 and recording chamber 1604 were modified to reduce chamber volume and perfusion times, which can increase the success rate of pharmacology experiments. A laser-guided positioning system can be used to achieve efficient alignment of target cells with the US beam focus. With system 1600, the variance of the ultrasound stimulation threshold for current activation was reduced.

For purpose of illustration and not limitation, pharmacological agents and ion substitution can be used to establish ion selectivity of ultrasound stimulated conductances in cell lines. For example, and as embodied herein, the in vitro ultrasound systems described herein were configured to enhance the reliability and efficiency of ultrasound stimulation of mammalian cells. As embodied herein, the ultrasound transducer mount 1602 and the recording chamber 1604 were configured to reduce chamber volume and perfusion times, which can increase the success rate in pharmacology applications. A laser-guided positioning system 1608 can achieve efficient alignment of target cells with the ultrasound beam focus. With this system, the variance of the ultrasound stimulation threshold for current activation was reduced.

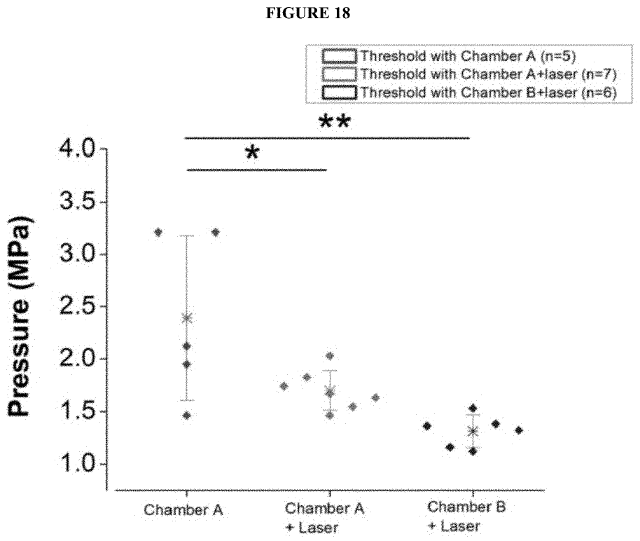

Ultrasound stimulation systems (system 400, also referred to as “Chamber A”), as described herein, includes an in vitro recording chamber 404 and a fixed ultrasound transducer mount 402. As described herein, system 400 can be used to stimulate cells plated near the center of the coverslip, where stimulation was applied by an ultrasound transducer. The precise focal area can be difficult to identify, and as such, only a small portion of cells plated near the center of the Mylar coverslip can be targeted with ultrasound sonication. For example and without limitation, and as embodied herein, to improve alignment of the ultrasound beam focus with target cells, in system 1600, a laser module 1608 (e.g., Quarton Inc. VLM-650-01) was integrated into the transducer mount, in a confocal arrangement (referred to as “Chamber A+laser”), as shown for example in FIG. 16A. As embodied herein, the laser beam illuminates a spot on the Mylar coverslip aligned with the ultrasound beam focus. For purpose of illustration and not limitation, as embodied herein, to confirm that the laser is aligned with the ultrasound beam focus, high intensity ultrasound was pulsed to physically etch the focal area on the Mylar coverslip, as shown for example on the top of FIG. 16B. As shown in FIG. 16B on the bottom, the laser beam directly aligned with the etched focal area, indicating successful targeting. For purpose of illustration and comparison, and not limitation, to show improved reproducibility of ultrasound stimulation, the ultrasound stimulation thresholds between Chamber A and Chamber A+laser were compared. With reference to FIG. 18, compared to Chamber A, cells stimulated in Chamber A+laser had a lower ultrasound threshold for activation and variance (variance test: ANOVA on absolute value of residuals, Bonferroni post-hoc, P=0.0004). As such, the addition of the laser to Chamber A in system 1600 reduced the variance of the ultrasound stimulation thresholds.

For purpose of illustration and not limitation, as embodied herein, characteristics of the Chamber A+laser were identified. The fixed position of the ultrasound transducer can affect the number of cells that can be targeted with the ultrasound beam. Additionally, the bath volume (˜50 ml) of the Chamber A+laser configuration can reduce the efficiency of solution exchange to perform pharmacological experiments. As such, with reference to FIG. 17A, system 1700 (also referred to herein as “Chamber B+laser”) further modifies system 1600 by separating the ultrasound transducer mount 1702 from the recording chamber 1704 and mounting it to a motorized 3D-manipulator 1710. Additionally, as embodied herein, the size of the recording chamber 1704 is reduced from about 50 mL to 3-4 mL.

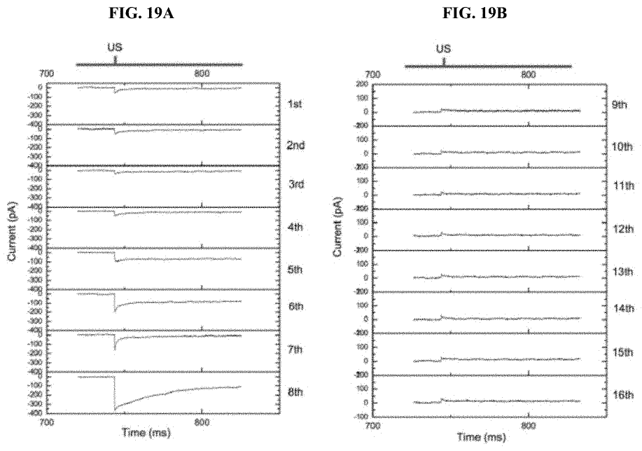

For example and not limitation, as embodied herein, in system 1700, the ultrasound adapter 1702, with a confocally mounted laser 1708, can be positioned independent of the recording chamber 1704. In this manner, each particular cell on the Mylar coverslip can be targeted, greatly increasing the efficiency of the system. Additionally, as embodied herein, the reduced size of the recording chamber 1704 allows for more rapid solution exchange for pharmacological applications. As such, as embodied herein, any cell visible under the microscope can be stimulated by the ultrasound transducer of system 1700. For purpose of illustration and comparison, and not limitation, with reference to FIG. 18, cells stimulated in Chamber B+laser had the lowest ultrasound threshold for activation and showed the least variance (variance test: ANOVA on absolute value of residuals, Bonferroni post-hoc, P=0.0003). As such, the two-component chamber of system 1700, with a confocally aligned laser targeting system (Chamber B+laser), substantially reduces the mean threshold and variance for ultrasound activation. FIG. 19 shows an example of the ultrasound-induced responses following 16 consecutive stimuli using system 1700, indicating reduced rundown or damage by ultrasound. The chamber configuration of system 1700 thus allows for a further improved ultrasound stimulation system for in vitro electrophysiological recordings.

For purpose of illustration and not limitation, as embodied herein, system 1700 can be used to measure the pharmacological profile of the ultrasound-induced currents in HEK cells. Ruthenium red (RR) can be used to block non-selective cation channels, including the mechanically activated ion channels Piezol, Piezo2, as well as some TRP channels (TRPA1, TRPV3, TRPV4, TRPV5, TRPV6). As embodied herein, RR (10 μM) can block ultrasound-induced whole-cell currents in HEK cells. For example and not limitation, as embodied herein, each cell was stimulated with ultrasound five times at both −90 mV (or −70 mV) and +30 mV (or +50 mV) alternatively. The same stimulation was applied 10 minutes after RR wash-in and 10 minutes after the RR washout, respectively. The first three responses, which usually showed higher reproducibility, were used to calculate the mean amplitude of the responses in each condition.

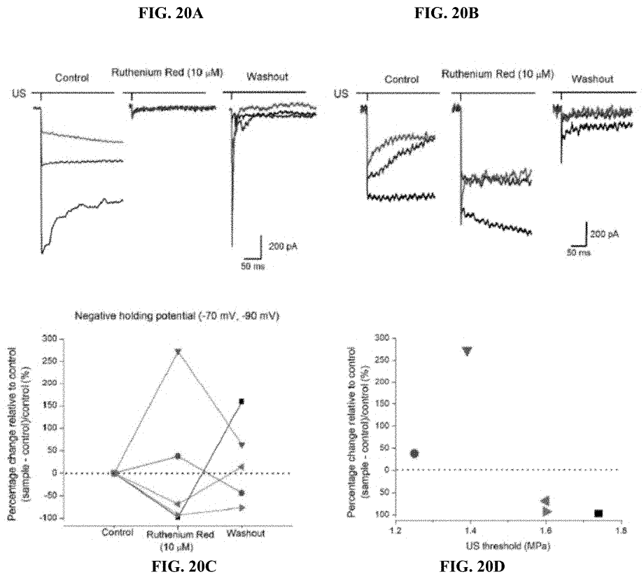

With reference to FIGS. 20A-20D, cells that showed at least partial recovery following washout were included for analysis (N=5). As embodied herein, RR showed varying effects on ultrasound-induced currents in HEK cells. Three cells showed reduced US-evoked currents, as illustrated in FIGS. 20A and 20C, whereas two cells showed enhanced US-induced currents in the presence of RR, as illustrated in FIGS. 20B and 20C. As embodied herein, the cells that showed reduced US-induced responses in the presence of RR had higher US activation thresholds (1.6-1.74 MPa) as compared to those showing enhanced responses in the presence of RR (1.25-1.37 MPa), as shown for example in FIG. 20D. As such, two populations of HEK cells or channels have different US activation thresholds and sensitivity to RR: 1) HEK cells of channels with high US activation thresholds are more sensitive to pharmacological block with RR, while 2) HEK cells or channels with low US activation thresholds are less sensitive to RR. Thus, although varying effects of RR on US-induced peak currents were observed across the total recorded HEK cell population, as shown for example in FIG. 20C, a putative cluster of HEK cells that that are sensitive to RR block were shown.

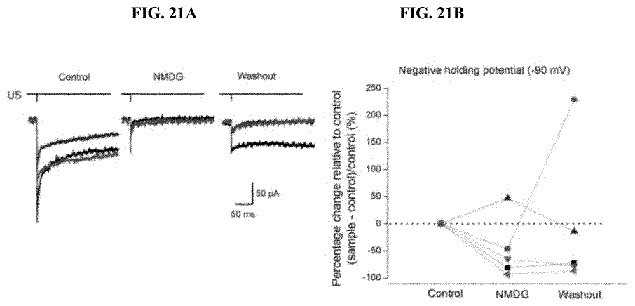

Additionally or alternatively, and as embodied herein, an extracellular cation, such as sodium, can be used to mediate US-induced inward currents. For example and not limitation, as embodied herein, cells were stimulated with US, first in Ringers solution (with sodium), then in an N-methyl-d-glucamine (NMDG)-based Ringer solution (without sodium). NMDG can be utilized at least in part because most mammalian cation channels can be impermeable to this large cation. As embodied herein, each cell was stimulated with US five times at −90 and +30 mV alternatively. The same stimuli were applied 10 minutes following NMDG replacement of sodium and 10 minutes following washout with Ringers (with sodium). Cells that did not show recovery at −90 mV following washout of NMDG were not included for analysis. The first three responses were used to calculate the mean amplitude of the responses in each condition. With reference to FIGS. 21A and 21B, in 4/5 cells, a reduction in US-activated currents with NMDG substitution was shown; however, in most cases, the effect was not reversible, as shown for example in FIG. 21B. This can be due at least in part because NMDG was not effectively washed out of chamber during the washout period (˜10 minutes). Additionally or alternatively, the lack of reversibility can indicate cellular rundown over the course of the stimulations.

According to other aspects of the disclosed subject matter, a research instrument to stimulate neurons for drug discovery is also provided. The research instrument can include any or all features of systems 200, 400, 1600 and 1700 described herein. Additionally, the research instrument can include high-throughput application of ultrasound to multi-chambered well plates or dishes, with simultaneous imaging or electrophysiological recording, to facilitate drug discovery.

In certain embodiments, the disclosed subject matter can provide precise control of nerve activity through non-invasive techniques. The non-invasive techniques can provide an application of FUS with certain parameters to control activity of the peripheral nervous system. FUS sonication (e.g., millisecond duration) can repeatedly evok action potentials in all peripheral neurons. For example, stimulus duration (e.g., 0.1-2.0 ms in 0.1-0.5 ms steps) and intensity (e.g., 11-743 W/cm2 in 25-60 W/cm2 steps) can be applied, with US frequency (e.g., about 1-5 MHz), and inter-stimulus interval (e.g., 0.1 ms-10 s).

In non-limiting embodiments, certain FUS parameters can excite all neuronal classes, including myelinated A fibers and unmyelinated C fibers. Peripheral neurons can be excited by FUS stimulation targeted to either skin receptive fields or peripheral nerve trunks. The disclosed FUS can elicit action potentials with millisecond latencies compared with electrical stimulation, through ion channel. For example, FUS thresholds can be increased in neurons lacking the mechanically gated channel Piezo2. In some embodiments, transcutaneous FUS can control peripheral nerve activity by engaging intrinsic mechanotransduction in neurons. The non-invasive techniques for PNS modulation can increase the safety and expand modulation application to various disease stages.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

### Example 1: Focused Ultrasound Excites Action Potentials in Mammalian Peripheral Neurons Through the Mechanically Activated Ion Channel Piezo2

This example provides systems and techniques for millisecond, high-intensity stimulation of sensory neurons with FUS to elicit action potentials in mechanosensory neuron.

Certain tissues including skin, heart, lung, gut, and immune organs, such as bone marrow, spleen and lymph nodes, can be innervated by neurons of the peripheral nervous system (PNS). These PNS neurons can serve both afferent functions, sending sensory information to the brain, and efferent roles, delivering neural signals to peripheral organs to tune their physiological outputs. For example, in the case of injury or infection, PNS neurons can represent a component of immune responses. The intersection between the PNS and organ systems thus can represent a target for therapeutic development. Certain peripheral neuromodulation devices are FDA approved or in clinical trials to treat wide-ranging diseases from depression to rheumatoid arthritis. These devices can use implanted electrodes, which can involve surgical procedures that inherent carry risk. Thus, non-invasive strategies to modulate PNS activity can be an appealing alternative to treat chronic diseases.

Focused ultrasound (FUS) can provide non-invasive neuromodulation of deep brain tissue. Stimulation of the CNS with ultrasound can elicit neuronal action potentials in hippocampal slices, non-invasively stimulate intact motor circuits, and display therapeutic potential for seizure disruption in mammals. Certain transdermal sonication can induce somatic sensations such as tactile, thermal, and pain, suggesting that ultrasound can activate sensory neurons. Non-invasive sonication of the mouse sciatic nerve can elicit muscle activity, indicating that FUS excites motor neurons. Certain sonication can evoke neural activity consistent with receptor- or action potentials.

In this example, reliable FUS parameters that excite action potentials in mammalian peripheral neurons in intact tissue were determined. Mechanosensory neurons of mouse dorsal root ganglia (DRG), whose peripheral axons, or afferents, can densely innervate skin and internal organs to convey sensory information to the CNS were assessed. Activation of primary sensory neurons can give rise to distinct sensations, including touch, pain, itch, warmth and cold. These distinct percepts can be initiated by an impressive array of somatosensory neuronal subtypes, including multiple classes of mechanoreceptors, thermoreceptors, and nociceptors (or pain-sensing neurons). Peripheral sensory neurons can be further classified based on neurophysiological properties, including conduction velocity (CV), receptive field, sensory threshold and firing pattern. The excitatory effects of FUS on these neurons assessed in intact mammalian tissue.

As embodied herein, millisecond, high-intensity stimulation of sensory neurons with FUS was sufficient to elicit action potentials in all mechanosensory neuron studied. These results define a parameter space to non-invasively excite sensory neurons in intact tissue, which can directly inform the development of neuromodulatory therapeutics.

In this example, as embodied herein, mice were maintained on a 12 h light/dark cycle, and food and water was provided ad libitum. Euthanasia was performed with isoflurane inhalation followed by cervical dislocation. Experiments were performed on 7-13 week old mice. The following strains were used in this study: female C57BL/6 (Jackson Labs), Cdx2Cre (27), and Piezo2fl/fl (41). For experiments involving tissue-specific deletion of Piezo2, genotypes that lacked either Cre or floxed Piezo2 alleles (Piezo2fl/fl or Piezo2fl/+) were designated as littermate controls, and Cdx2Cre; Piezo2fl/fl were experimental animals.

FUS was delivered with a commercial focused ultrasound transducer with a 3.57 MHz center frequency (35 mm focal depth; SU-107, Sonic Concepts). Driving signals were delivered by a function generator (33220A, Keysight Technologies) and amplified through a 150 W amplifier (A150, Electronics & Innovation). To calibrate the transducer (Table S4), beam plots were acquired using a fiber-optic hydrophone (HF0690, Onda). The transducer was mounted on a 3D motorized XYZ positioner (Bislide, Velmex). After locating the center of the ultrasound focus, 2D raster scans in both XY and XZ planes were acquired (100 cycle bursts and a 10 Hz pulse repetition frequency).

To deliver targeted FUS stimulation of neurons, we constructed a custom immersion cone, equipped with guide lasers (VLM-650-01 LPA, Quarton USA) to identify the ultrasound focus. The cone was filled with degassed water and the tip was sealed with a thin plastic membrane (CE0434, EMT Medical Co). Using the intersection of the lasers as a guide, the focus of the transducer was positioned with a 3D micromanipulator (MPC-200, Sutter Instrument) on the receptive field or the saphenous nerve trunk. To ensure continuous coupling of the transducer to the target, a small volume of bath solution was maintained between the tip of the immersion cone and the target surface.

FUS parameters employed were: stimulus duration (0.1-2.0 ms, 0.1-0.5 ms steps), intensity (11-743 W/cm2, 25-60 W/cm2 steps), US frequency (3.57 MHz), and inter-stimulus interval (5 s). Stimulus order was typically from short-to-long duration and low-to-high intensity. The latency of FUS-evoked action potentials was measured from the FUS trigger to action potential peak. FUS-thresholds were defined as the first sonication energy that generated action potentials in >50% of stimulus presentations.

Action potentials from teased nerve fibers were recorded after dissecting the mouse hindlimb skin and saphenous nerve according to published methods. Tissue was placed epidermis-side-up in a custom chamber and perfused with carbogen-buffered synthetic interstitial fluid (in mM: 108 NaCl, 3.5 KCl, 0.7 MgSO4, 26 NaHCO3, 1.7 NaH2PO4, 9.5 sodium gluconate, 5.5 glucose, 7.5 sucrose, and 1.5 CaCl2, saturated with 95% 02-5% CO2; pH 7.4) kept at 32° C. with a temperature controller (TC-344B, Warner Instruments). The nerve was kept in mineral oil in a recording chamber, teased, and placed onto a recording electrode connected with a reference electrode to a differential amplifier (model 1800, A-M Systems). The extracellular signal was digitized using a PowerLab 8/35 board (AD Instruments) and recorded using LabChart software (AD Instruments). Sampling frequencies were 20 kHz or 40 kHz.

Single units and their receptive fields were identified using mechanical search with a blunt glass probe. Once isolated, afferents were characterized based on mechanical threshold, receptive field characteristics, CV and adaptation properties to sustained mechanical stimuli. Mechanical threshold was measured by stimulating receptive fields with calibrated von Frey monofilaments. Mechanical thresholds were defined as the first von Frey monofilament that generated action potentials in >50% of stimulus presentations. Receptive fields and responses to hair movement were evaluated under stereomicroscopy, by deflecting individual hairs with fine forceps (Model SZX16; Olympus). CV was estimated based on electrical stimulation of receptive fields delivered from a pulse stimulator (Model 2100, A-M Systems). CV was calculated as the quotient of distance between the stimulus and recoding electrodes, and the latency of the action potential peak from the stimulus artifact. To assess adaptation properties, receptive fields were stimulated with a custom-built, computer controlled mechanical stimulator (tip diameter: 1.6 mm).

For experiments in Cdx2Cre;Piezo2fl/fl and littermate control mice, an electrical search was used to identify afferents. Electrical stimulation was delivered first near where the saphenous nerve inserts into the skin, and progressively more distal, to approximate receptive field locations. Once electrically-identified receptive field locations were established, mechanical thresholds, receptive field characteristics, CV and adaptation properties to sustained mechanical stimuli, were estimated as described above.

Mechanosensory afferents were classified into five subtypes based on physiological response properties: Aβ rapidly adapting (Aβ-RA), Aβ slowly adapting (Aβ-SA), D-hair mechanoreceptor (DH), A-fiber mechanonociceptor (AM), and C-fibers. Classification was performed based on criteria modified from ref.: AP-RA fibers, CV>˜10 m/s, no response to zig-zag hair movement, RA responses to 5-s mechanical stimulation; AP-SA fibers, CV>˜10 m/s, responded to touch dome indentation and/or hair movement, sustained responses to 5-s mechanical stimulation; DH fibers, CV≥1 m/s and ≤10 m/s, responses to zig-zag hair movement; AM fibers, CV≥1 m/s and ≤10 m/s, no response to hair movement, and SA responses to 5-s mechanical stimulation; C-fibers, CV<1 m/s.

Spike sorting and data analysis was performed in Matlab. Spikes were sorted based on the following parameters: positive peak amplitude, negative peak amplitude, positive peak rise time, spike width, and negative peak decay time. Sorted waveforms were then averaged to generate a template, which was then compared back to the sorted waveforms with correlation analysis. Spikes kept for further analysis had correlation coefficients of >0.97 in A-fibers and >0.85 in C-fibers.

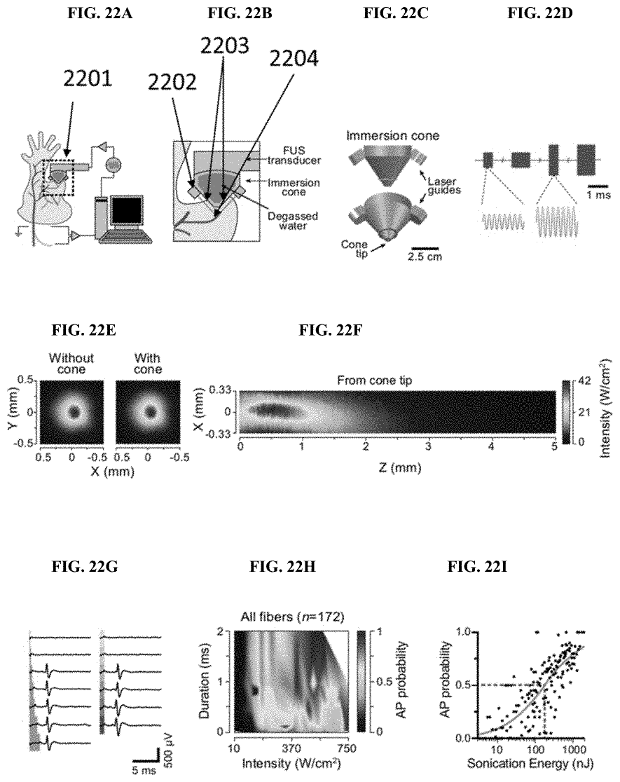

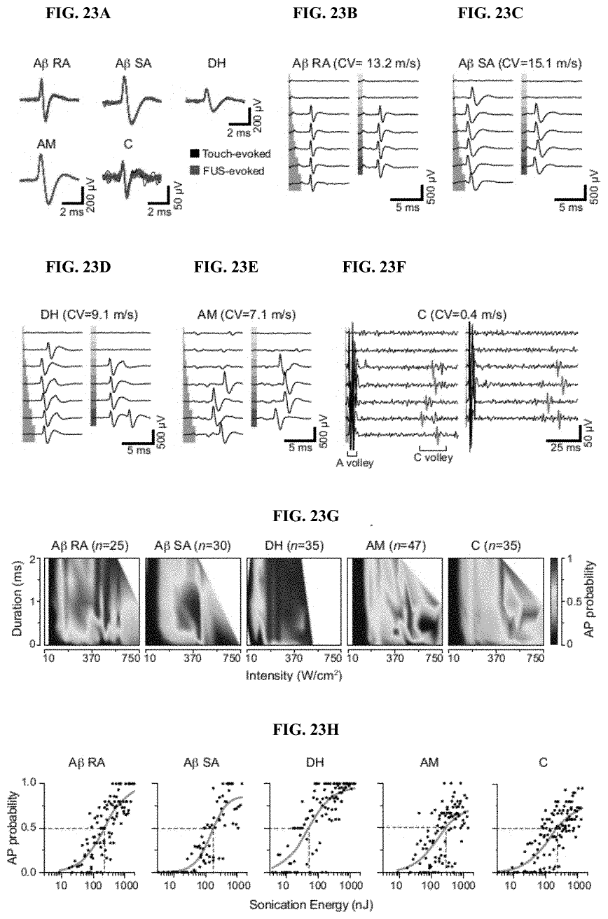

Action potential probability was calculated for each FUS parameter combination delivered to each recorded neuron. To generate aggregate parameter exploration data (FIGS. 22 and 23), action potential probabilities for each sampled FUS parameter combination were averaged across fibers. Given that the parameters delivered to each recorded fiber varied, only parameters that were delivered to at least two fibers were considered for further analysis. To generate continuous surface plots, probability data was interpolated with the “scatteredInterpolant” function using “linear” interpolation. Sonication energy was calculated from aggregate FUS parameter-probability datasets using following function:

Sonication Energy(J)=I×πr²×t

I=intensity (W/cm²);r=US focal radius (cm);t=sonication duration (s) (1)

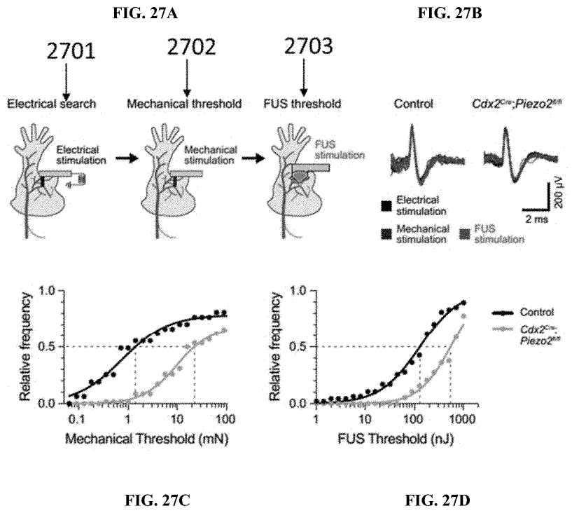

Sonication energy (FIGS. 22I and 23H) and cumulative response profiles (FIGS. 27C and 27D) were fit with the following dose-response function (note, the “bottom” variable is fixed to 0):

$$y = \frac{a}{1 + {10}^{( ( ( {log}_{10} b ) - x ) \times c )}} ; a = Top ; b = {EC}_{50} ; c = slope \qquad ( 2 )$$

Statistical analysis was performed in Matlab (MathWorks) and Prism (Graphpad). Statistical parameters are described in figure legends. Paired student's two-tailed t test was used to compare means of two normally distributed, paired groups. Wilcoxen Signed Rank Test was used to compare the medians of two non-parametric groups. Non-parametric data with three or more groups were analyzed using the Kruskal-Wallis test. Correlations between non-parametric groups was computed using Spearman's rank-order correlation. The normality of population data was assessed using the Kolmogorov-Smirnov test with Dallal-Wilkinson-Lilliefors P values, with P<0.05 indicating non-normality. Differences were identified if P<0.05.

An experimental paradigm using mouse ex vivo skin-nerve preparations that enables simultaneous FUS stimulation and electrophysiological recordings from individual peripheral neurons was developed (FIG. 22A). To accomplish targeted sonication of sensory neurons, an immersion cone 2202 was equipped with two lasers 2203 that intersect at the center of the ultrasound focus 2204 (FIGS. 22B and 22C). The immersion cone thus provides, among other features, coupling of the ultrasound beam to the target tissue with degassed water, and laser guided positioning of the FUS focus to tissues of interest. FUS was generated with a transducer controlled by 3.57 MHz sine waves delivered from a function generator (FIG. 22D). The resulting ultrasound field had a focal diameter (full-width at half maximum) of 0.33 mm and a focal length of 1.16 mm from cone tip (FIG. 22E-22F).

Mechanosensory neurons that innervate skin and that initiate senses such as touch and mechanical pain were analyzed. After establishing an extracellular recording from teased nerve fibers, a neuron's receptive field (the area of skin it innervates) was identified by gently pressing the skin with a blunt rod. Next, the receptive field was sonicated with laser-guided FUS. To identify efficient and reliable FUS protocols, neurons were sequentially stimulated with varying combinations of FUS parameters. Stimulus duration (0.1-2.0 ms in 0.1-0.5 ms steps) and intensity (11-743 W/cm2 in 25-60 W/cm2 steps) were varied, while US frequency (3.57 MHz), and inter-stimulus interval (5 s) remained fixed. Each FUS parameter set was presented 4 10 times, and action potential probability was estimated as the fraction of stimuli that elicited an action potential. FUS stimulation within this range had negligible thermal effects (<1° C.; FUS parameters: 2 ms, 743 W/cm2).

More than 100 FUS parameter combinations in mechanosensory neurons were tested. High-intensity, millisecond sonication with FUS reliably excited single action potentials (FIG. 22G). All recorded sensory neurons were excited by sonication (n=172/172). Over the ranges tested, increasing either sonication duration or intensity increased action potential probability (FIG. 22H). Indeed, total sonication energy, which is proportional to the product of intensity and stimulus duration, showed a strong positive correlation with action potential probability (FIG. 22I; R=0.81, P<0.0001, Spearman's correlation, Max_{AP}=1.0, slope=0.79, EC₅₀=186, E_{50%Prob}=186, R²=0.64, and E_{50%Prob}=red dot).

These data illustrate an FUS parameter space to excite peripheral neurons, and indicate that the primary driver of FUS-evoked action potentials is the amount of energy delivered.

Mechanosensory neurons that serve different roles in vivo can be functionally classified ex vivo based on their electrophysiological properties. Aβ rapidly adapting (Aβ-RA) and Aβ slowly adapting (Aβ-SA) fibers are myelinated, fast-conducting fibers that encode tactile information. D-hair (DH) mechanoreceptors are intermediately conducting, Aβ fibers that report hair movement. Noxious mechanical stimuli are encoded by A-fiber mechanonociceptor (AM) and most C-fibers, which have unmyelinated axons. Thus, these exemplary classes of sensory neurons were tested whether they respond differentially to FUS parameter combinations by partitioning our neuronal dataset into these five classes, as described herein: Aβ-RA (n=25), Aβ-SA; (n=30), DH (n=35), AM (n=47), and C-fibers (n=35; FIGS. 23A-23F).

All neuronal classes examined were excited by sonication. Comparison of the two-dimensional FUS parameter space by class illustrated that short (˜0.75 ms), high-intensity (350-500 W/cm2) sonication was highly effective in evoking action potentials across all classes (FIG. 23G). To directly compare FUS sensitivity among classes, total sonication energy was assessed, which positively correlated with action potential probability in all fiber types (FIG. 23H; Aβ RA, R=0.81; Aβ SA, R=0.86; DH, R=0.77; AM, R=0.62; C, R=0.76; P<0.0001, Spearman's correlation). Pseudocolor axis represent action potential probability (parameter sets: AβRA, n=101; AβSA, n=76; DH, n=100; AM, n=101; C, n=125). For each fiber class, data were fit with a stimulus-response relation to estimate the maximal action potential probability (MaxAP) and the energy at which the probability of firing was 50% (E_{50%prob}). DH neurons, which are ultrasensitive to light touch, were more likely to be excited by US than any other class (Max_{AP}=0.97, E_{50%prob}=50 nJ). By contrast, AM fibers, which have higher mechanical thresholds (Max_{AP}=0.77, E_{50%prob}=280 nJ), were less excitable overall. AO and DH fibers had a higher Max_{AP} compared with AM and C-fibers; therefore, low-threshold mechanoreceptors can follow FUS stimulation with improved reliability compared to nociceptors over this range. Data are displayed as the mean action potential probability at each sonication energy sampled. Together, these data define a range of FUS parameters (˜0.75 ms, 350-500 W/cm2) capable of exciting all mechanosensory neurons.

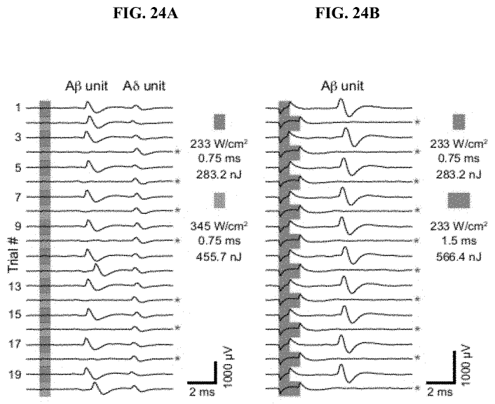

Certain fibers displayed non-monotonic tuning in their probability-response profiles. In these neurons, action potential probability first increased and then decreased with progressively higher energy FUS stimulation (high-intensity and/or long sonication duration). Indeed, in these neurons alternating optimal FUS stimulation parameters with supra-optimal parameters enabled selective control of action potential generation (FIG. 24). The failure to elicit action potentials with supra-optimal FUS stimulation does not represent damage, as optimal stimulation consistently elicited action potentials within 5 s of supra-optimal stimulation. Only a fraction of total neurons with supra-optimal FUS stimulation displayed this type of response to high-energy FUS (high-intensity, n=20/164; long-duration, n=22/136), which suggest that intrinsic properties of neural subsets can facilitate this tuning phenomenon. Together, these data provide an exemplary strategy to selectively activate subsets of sensory neurons, which can be used for therapeutic applications.

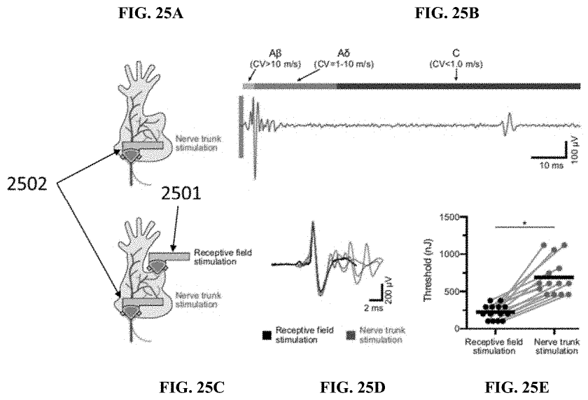

One therapeutic application of FUS neuromodulation is the non-invasive stimulation of nerves, such as the vagus nerve, to manipulate neurohumoral reflexes. Such a device can provide stimulation of nerve trunks rather than receptive fields. Thus, FUS sonication of peripheral nerve trunks was tested to assess whether the sonication can evoke action potentials. The saphenous nerve trunk with FUS was tested, which elicited compound action potentials composed of AP, Aβ and C fiber activity (FIGS. 25A and 25B). FUS stimulation of receptive fields 2501 was compared with nerve trunks 2502 for individual mechanosensory neurons (FIG. 25C). Mechanosensory afferents were first identified with manual exploration of receptive fields 2501 with a blunt glass probe. Once identified and characterized, afferents were sequentially stimulated with FUS targeted to nerve trunks 2502 and to receptive fields 2501. Most fibers that responded to FUS receptive field stimulation were activated by FUS sonication of nerve trunks (n=14/15; FIG. 25D). The 50% threshold to activate action potentials was threefold higher in nerve trunk stimulation compared with receptive field stimulation (FIG. 25C). As such, neurons can be targeted at either receptive fields or along nerve trunks, and effective sonication ranges can be defined for activation in both stimulus paradigms. FIG. 25D shows comparison of FUS-evoked action potentials elicited by receptive field stimulation (black) and nerve trunk stimulation (magenta). FIG. 25E shows comparison of sonication energy thresholds (e.g., greater than 50% probability of eliciting an action potential) from FUS stimulation targeted to receptive fields or nerve trunks from the same fiber. Gray lines represent thresholds from the same fiber (P<0.0001, two-tailed paired t-test, n=14 fibers).

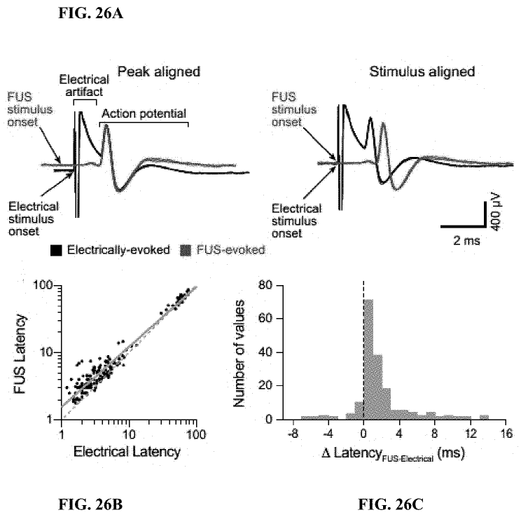

Certain FDA-approved neuromodulation devices employ electrical nerve stimulation. These technologies can depolarize neurons to activate voltage-gated sodium channels (NaVs), which can faithfully and rapidly trigger action potentials. FUS parameters that reliably activate one-to-one action potentials can be used to assess how FUS stimulation compares to electrical stimulation in terms of speed. Analysis of peak-aligned waveforms for individual neural responses showed that electrically evoked spike waveforms were similar to those elicited by FUS for all fibers examined. As such, the same fibers are activated by both stimuli (FIG. 26A, left). When waveforms were aligned by stimulus onset, FUS-evoked action potential latencies were ˜1 ms longer than those measured from electrical stimulation (FIG. 26A, right). Latencies for FUS- and electrically evoked action potentials were positively correlated (A-fibers, R=0.80, P<0.0001; C-fibers, R=0.85, P<0.0001; Spearman's correlation) and were well fit by linear regression with a positive intercept and a shift towards the FUS axis (FIG. 26B; slope, 0.89 ms; y-intercept, 0.20 ms; R2=0.95). Across the population, the difference between FUS and electrical latencies (Δ Latency) measured from the same fiber was in the millisecond range (median=0.9 ms, interquartile range=1.8 ms, n=172; FIG. 26C). As such, the molecular mechanisms that underlie FUS-evoked stimulation of peripheral neurons can activate within milliseconds, which is consistent with neuron-autonomous, ion-channel mechanisms.

FUS can excite action potentials either by directly activating voltage-gated sodium channels, or by activating upstream sensory ion channels that depolarize neurons to action potential threshold. FUS-evoked action potential latencies can be consistently ˜1 ms longer than electrical stimulation, and as such, FUS activates fast sensory ion channels, such as mechanically gated ion channels. Mechanically gated ion channels encoded by Piezo2 can be used as a mechanotransduction mechanism in mammalian A-fiber mechanosensory neurons. Thus, activation of peripheral neurons with sonication using Piezo2 was investigated. Cdx2Cre;Piezo2fl/fl mice were generated, which harbor a deletion of Piezo2 in caudal tissues including peripheral neurons. Mechanosensitivity is reduced in A-fiber mechanosensory neurons lacking functional Piezo2; thus, an electrical search 2701 was used to identify A-fiber responses from Cdx2Cre;Piezo2fl/fl and control genotypes, and then thresholds were measured for mechanically 2702 or FUS-evoked action potentials 2703 (FIGS. 27A and 27B). To compare these thresholds across genotypes, cumulative response profiles were assessed for each stimulus. Data were then fit with stimulus-response relations to estimate the stimulus magnitude at which the 50% of fibers responded (E_{50%Respond}). Peripheral neurons from Cdx2Cre;Piezo2fl/fl mice displayed a marked increase in mechanical threshold (E50=22.0 mN) compared with control genotypes (E50=1.4 mN; FIG. 27C). Few fibers showed mechanical thresholds in the innocuous range (≤4 mN), confirming published reports that Piezo2 channels can be used for neural responses to touch stimuli. Likewise, afferents from Cdx2Cre;Piezo2fl/fl mice displayed a dramatic reduction in sensitivity to FUS sonication (E_{50%Respond}=535.8 nJ) compared with controls (E_{50%Respond}=128.2 nJ). Together, these data identify Piezo2 as a molecular effector of FUS neuromodulation in mammalian peripheral neurons.

As embodied herein, US sonication can directly and robustly evoke action potentials from individual neurons in the mammalian PNS. Millisecond, high-intensity (350-500 W/cm2) sonication of neuronal receptive fields was sufficient to elicit action potentials in both myelinated (Aβ and Aβ) and unmyelinated (C) fibers. Action potentials followed FUS sonication in a one-to-one manner, demonstrating that FUS can allow tight temporal control over neuronal activity in the PNS. FUS stimulation of nerve trunks excited action potentials effectively at higher sonication energies. Effective parameters for non-invasive excitation of peripheral nerves with ultrasound in intact tissue are provided, which can be useful for the development of ultrasound-based therapeutics.

There was no significant increase in temperature with maximal FUS stimulus parameters. As such, ultrasound stimulation can occur under conditions that minimally heat tissues. Thus, the thermal effects of sonication under the disclosed experimental conditions can be minimized or reduced, and robust and repeatable neuronal activation can be provided.

The sonication can induce mechanical effects on neural tissue, such as radiation force, membrane oscillation or cavitation, resulting in the activation of ion channels and action potential generation. Radiation force can activate mechanosensitive MEC-4 channels in C. elegans. FUS can activate mammalian mechanosensitive ion channels, such as the Piezo family of proteins. Certain low-threshold mechanoreceptors were more sensitive to FUS stimulation than nociceptors in this study. FUS can initiate the opening of other ion channels, such as voltage gated sodium channels, which can display mechanical gating. In hippocampal slices, ultrasound can stimulate action potentials through mechanical activation of voltage-gated sodium and calcium channels. FUS can also activate voltage-gated potassium channels, some of which are mechanosensitive, such as the TRAAK and TREK channels. Activation of potassium channels, which results in decreased neuronal excitability, can illustrate that the probability of FUS-evoked action potentials decreases at higher FUS stimulus intensities.

US sonication to human skin can initiate somatic sensations such as warmth, pain, and pressure; however, the potential therapeutic applications of neuromodulation of peripheral nerve activity can extend beyond sensory modulation. One such application can be non-invasive modulation of the neural reflex arc to treat chronic disease. The neural reflex arc can be composed of peripheral afferent neurons that signal to the CNS, and efferent neurons that send regulatory signals to virtually all peripheral tissues. Stimulation of the vagus nerve, which can be composed of both afferent and efferent neurons, is an FDA approved intervention for epilepsy and treatment resistant-depression, and can be used for diseases such as rheumatoid arthritis, systemic lupus erythematosus, Chron's disease, and hypertension. Certain therapeutics for vagus nerve stimulation can involve surgically implanted electrodes, which can result in significant complications. The disclosed data provides a non-invasive, ultrasound-based device that can avoid surgical implantation of electrodes in vagus nerve targeting therapeutics. As embodied herein, the axons of peripheral neurons within nerve trunks were reliably excited by FUS stimulation.

Neuronal subtypes showed different sensitivities to FUS stimulation. DH neurons, which are highly sensitive, low-threshold mechanoreceptors that innervate hair follicles, were the neurons most sensitive to FUS stimulation. AM neurons, which are nociceptors that encode pain, received greater sonication energies to activate. Certain neurons displayed non-monotonic dose response relationships to FUS stimulation, and were suppressed at larger stimulation magnitudes. As such, as described herein, certain FUS parameter combinations can efficiently and selectively activate, or suppress, action potential firing in subsets of neurons. Thus, FUS neuromodulation can improve therapy with the ability to selectively target neurons within mixed nerves. The vagus nerve, for example, contains afferent and efferent neurons that innervate the majority of visceral tissues, such as the heart, lung and gut, as well as immune organs. Pathologies of specific organs, or organ systems, can utilize non-invasive and selective neuromodulation of the vagal subset of neurons that innervate them.

What is claimed is:

1. A system for modulating one or more neurons using focused ultrasound (FUS), comprising:
a transducer mount;
a recording chamber disposed at an angle relative the transducer mount and configured to contain the one or more neurons within the recording chamber;
an ultrasound transducer disposed on the transducer mount to provide an ultrasound stimulus having one or more ultrasound parameters to the one or more neurons; and
a processor, coupled to the ultrasound transducer, configured to adjust the one or more ultrasound parameters to produce one or more action potentials from the one or more neurons in response to the ultrasound stimulus, the one or more action potentials corresponding to one or more of a pain or sensation response, a pain or sensation suppression, or neural control of organ function induced by the one or more neurons.

2. The system of claim 1, further comprising an electrical sensor coupled to the processor, the processor further configured to receive the one or more action potentials from the electrical sensor.

3. The system of claim 1, further comprising a thermal sensor coupled to the processor, the processor further configured to measure a temperature of the one or more neurons during modulation.

4. The system of claim 1, wherein the angle of the recording chamber is about 25 degrees relative to the transducer.

5. The system of claim 1, wherein the one or more ultrasound parameters includes an ultrasound pressure of 3.2-5 MPa.

6. The system of claim 1, wherein the one or more ultrasound parameters includes a center frequency of about 3.57 MHz or 3.1 MHz.

7. The system of claim 1, wherein the one or more neurons comprises a sensory neuron.

8. The system of claim 1, wherein the one or more neurons comprises a motor neuron.

9. The system of claim 1, wherein the one or more neurons is disposed within the recording chamber ex vivo.

10. The system of claim 1, wherein the one or more neurons is disposed within the recording chamber in vitro.

11. A method for modulating one or more neurons using focused ultrasound (FUS), comprising:
providing a recording chamber at an angle relative a transducer mount;
inserting the one or more neurons within the recording chamber;
providing an ultrasound transducer disposed within the transducer mount to provide an ultrasound stimulus having one or more ultrasound parameters to the one or more neurons; and
adjusting the one or more ultrasound parameters, using a processor coupled to the ultrasound transducer, to produce one or more action potentials from the one or more neurons in response to the ultrasound stimulus, the one or more action potentials corresponding to one or more of a pain or sensation response, a pain or sensation suppression, or neural control of organ function induced by the one or more neurons.

12. The method of claim 11, further comprising an electrical sensor coupled to the processor, the processor further configured to receive the one or more action potentials from the electrical sensor.

13. The method of claim 11, further comprising a thermal sensor coupled to the processor, the processor further configured to measure a temperature of the one or more neurons during modulation.

14. The method of claim 11, wherein the angle of the recording chamber is about 25 degrees relative to the transducer.

15. The method of claim 11, wherein the one or more ultrasound parameters includes an ultrasound pressure of 3.2-5 MPa.

16. The method of claim 11, wherein the one or more ultrasound parameters includes a center frequency of about 3.57 MHz or 3.1 MHz.

17. The method of claim 11, wherein the one or more neurons comprises a sensory neuron.

18. The method of claim 11, wherein the one or more neurons comprises a motor neuron.

19. The method of claim 11, wherein the one or more neurons is disposed within the recording chamber ex vivo.

20. The method of claim 11, wherein the one or more neurons is disposed within the recording chamber in vitro.
